(19) United States
(12) Patent Application Publication
(10) Pub. No.: US 2013/0071482 A1
(43) Pub. Date: Mar. 21, 2013

(54) BLOCK COPOLYMER CROSS-LINKED NANOASSEMBLIES AS MODULAR DELIVERY VEHICLES

(71) Applicant: The University of Kentucky Research Foundation, Lexington, KY (US)

(72) Inventors: Younsoo Bae, Lexington, KY (US); Juergen Rohr, Georgetown, KY (US); Daniel Franklin Scott, Lexington, KY (US)

(73) Assignee: THE UNIVERSITY OF KENTUCKY RESEARCH FOUNDATION, Lexington, KY (US)

(21) Appl. No.: 13/623,470

(22) Filed: Sep. 20, 2012

Related U.S. Application Data
(60) Provisional application No. 61/536,764, filed on Sep. 20, 2011.

(51) Int. Cl. A61K 9/14; B01J 13/18; A61K 31/787; A61P 35/00; C08G 63/00; A61K 31/785; B82Y 40/00; B82Y 5/00

(52) U.S. Cl. 424/497; 525/437; 264/4.7; 424/78.3; 424/78.17; 977/773; 977/906; 977/896

(57) ABSTRACT

A nanoassembly includes a core protected by a biocompatible shell. The nanoassembly includes a plurality of block copolymers including drug-binding linkers and block copolymer cross-linkers. A first active agent is covalently conjugated to the plurality of block copolymers and a second active agent is physically entrapped in the core.

This utility patent application claims the benefit of priority in U.S. Provisional Patent Application Ser. No. 61/536,764 filed on Sep. 20, 2011, the entirety of the disclosure of which is incorporated herein by reference.

### TECHNICAL FIELD

This document relates generally to the field of drug/gene delivery using biocompatible nanoparticles and, more particularly, to block copolymer cross-linked nanoassemblies including a core protected by a biocompatible shell and methods for their production.

### BACKGROUND

Biocompatible nanoparticles, typically 5 to 200 nanometers (nm) in diameter, have garnered increasing attention as drug carriers in recent biomedical applications. Nanoparticles can circulate through blood vessels and accumulate preferentially in disease tissues that enhance permeation and retention of large molecules (>1,000 atoms), such as inflammatory tissues and cancerous tumors. One important property of these nanoparticle drug carriers is capability to control drug release rates, by time or in response to stimuli, and thus maintaining drug concentrations at therapeutic dose levels in targeted sites in vivo to maximize therapeutic efficacy and minimize toxicity. Drug release rates, however, are still difficult to control precisely in vivo partially because drug carriers often change particle size, stability, biocompatibility and other physicochemical properties, which affect their drug release patterns and result in undesirable plasma protein adsorption, macrophagic uptake, and non-specific tissue distribution. Therefore, it is important to develop an easy and efficient method to fine-tune drug release rates in vivo for nanoparticle drug carriers.

This document relates to the development of block copolymer cross-linked nanoassemblies which both covalently conjugate and physically entrap one or more active agents. Such nanoassemblies allow one to fine-tune active agent release rates in vivo in order to maximize diagnostic, imaging and/or therapeutic efficacy at the target site. In addition, a simple and effective method is described for constructing uniform and versatile biocompatible nanoassemblies for controlled entrapment and release of various guest molecules (e.g. anticancer drugs and imaging agents) through a simple synthesis process.

### SUMMARY

In accordance with the purposes described herein, a nanoassembly is provided that includes a core protected by a biocompatible shell. The nanoassembly comprises a plurality of block copolymers including drug-binding linkers and block copolymer cross-linkers, a first active agent covalently conjugated to the plurality of block copolymers and a second active agent physically entrapped in the core by the plurality of block copolymers. The block copolymers include two segments: one that is hydrophilic and one that is hydrophobic, or one that is biologically inert and one that is chemically modifiable.

In one useful embodiment the nanoassembly has a diameter of 5 nm to 200 nm. In one useful embodiment the nanoassembly has a cross-linking yield of 1% to 50%. In one useful embodiment the nanoassembly has a drug loading of between 1% to 60% by weight. In one useful embodiment the drug-binding linkers include permanent linkers and degradable linkers. In one useful embodiment the cross-linkers include permanent linkers and degradable linkers.

More specifically, the drug-binding linkers and/or the cross-linkers are selected from a group consisting of (1) an aliphatic compound with amino, carboxyl, hydroxyl, ketone or thiol groups and cross-linking formed through amide, ester, carbamate, imine, hydrazone, and disulfide bonds, (2) an aromatic compound with amino, carboxyl, hydroxyl, ketone or thiol groups and cross-linking formed through amide, ester, carbamate, imine, hydrazone, and disulfide bonds and (3) mixtures thereof.

In one embodiment the cross-linkers include pH degradable bonds. In one useful embodiment the cross-linkers include light degradable bonds. In one useful embodiment the cross-linkers include heat degradable bonds. In one useful embodiment the cross-linkers include bonds degradable by enzymatic activity. In one useful embodiment the cross-linkers include bonds degradable by hydrolysis. In one useful embodiment the cross-linkers include bonds degradable by redox reactions.

Still further, the first active agent is selected from a group consisting of a diagnostic agent, an imaging agent, a therapeutic agent, multiple diagnostic agents, multiple imaging agents, multiple therapeutic agents and mixtures thereof. The second active agent is selected from a group consisting of a diagnostic agent, an imaging agent, a therapeutic agent, multiple diagnostic agents, multiple imaging agents, multiple therapeutic agents and mixtures thereof. In one embodiment the first active agent and second active agent are the same diagnostic agent(s), imaging agent(s), therapeutic agent(s) and mixtures thereof. In another embodiment the first active agent and second active agent are different. Further at least one of the first active agent and second active agent is selected from a group consisting of a small molecule having less than 1,000 atoms, a large molecule having at least 1,000 atoms, a peptide, plasmid DNA, siRNA, a fluorescent dye, a contrast agent and mixtures thereof. In addition in one embodiment the first active agent and second active agent include at least one hydrophobic agent and at least one hydrophilic agent in a single nanoassembly.

In accordance with an additional aspect, this document also relates to a method of making a biocompatible nanoassembly. That method comprises cross-linking block copolymers including drug-binding linkers and block-copolymer cross-linkers, covalently binding a first active agent to the block copolymers, and physically entrapping a second active agent in the cross-linked block copolymers. The method further includes providing a plurality of block copolymers, with drug-binding linkers and block copolymer cross-linkers. In addition the method includes dissolving (a) block copolymers, with drug-binding linkers and block copolymer cross-linkers, (b) a first active agent and (c) a second active agent together in a solvent and simultaneously completing the cross-linking, covalent binding and physical entrapping. Still further the method includes simultaneously completing the cross-linking, covalent binding and physical entrapping while providing a cross-linking yield of between 1% and 50%, a drug loading of between 1% and 60% by weight and maintaining a nanoassembly having a diameter of 5 nm to 200 nm.

In the following description there are shown and described preferred embodiments of nanoassemblies and methods for producing those nanoassemblies. As it will be realized, the nanoassemblies and their methods of production are capable of other different embodiments and their several details are capable of modification in various, obvious aspects. Accordingly, the drawings and descriptions should be regarded as illustrative in nature and not as restrictive.

### BRIEF DESCRIPTION OF THE DRAWINGS

The accompanying drawings incorporated herein and forming a part of the specification, illustrate several aspects of the nanoassemblies and together with the description serve to explain certain principles thereof. In the drawings:

FIG. 1 is a schematic illustration showing four different types of biocompatible nanoassemblies;

FIG. 2 is a schematical illustration of a nanoassembly including a plurality of block copolymers cross-linked together with a first active agent covalently conjugated to the plurality of block copolymers and a second active agent physically entrapped in the core of the nanoassembly by the blocked copolymers;

FIG. 3 is a bar graph illustrating particle size of (1) self-assembled nanoassemblies (SNA), before and after entrapping a model anticancer drug doxorubicin (DOX) physically, (2) self-assembled nanoassemblies before and after entrapping DOX covalently bound to the block copolymers forming the nanoassemblies (Hyd-SNA), (3) cross-linked nanoassemblies (CNA) and (4) cross-linked nanoassemblies before and after entrapping DOX covalently bound to the block copolymers forming the nanoassemblies (Hyd-CNA);

FIGS. 4a-4d graphically illustrate drug/active agent release patterns for SNA, Hyd-SNA, CNA and Hyd-CNA nanoassemblies as illustrated in FIG. 1; and

FIGS. 5a and 5b graphically illustrate dose response curves of drug-loaded nanoassemblies by in-vitro cytotoxicity assays using free DOX and empty nanoassemblies as controls.

Reference will now be made in detail to the present preferred embodiments of the nanoassemblies, examples of which are illustrated in the accompanying drawings.

### DETAILED DESCRIPTION OF THE INVENTION

A “block copolymer” refers to a polymer with repeating units of one type adjacent to each other in a linear manner to form a block, which is linked, for example, through a covalent bond to a second block made up of repeating units of a second type, which are adjacent to one another in a linear manner to form a second block of the block copolymer. Block copolymers referred to in this document may have a natural, synthetic or semi-synthetic polymer backbone (e.g. polyester, polyether, polyamide, polypeptide, fatty acid, and combinations thereof). Each polymer chain may have a molecular weight of 1-50 kDa. The block copolymers include a hydrophilic segment and a hydrophobic segment, or one that is biologically inert and one that is chemically modifiable. Block copolymers useful in the present invention include, but are not limited to protected form block copolymers, anionic form block copolymers, cationic form block copolymers, cross-linked form block copolymers, drug-binding linker form block copolymers, drug-conjugated form block copolymer and mixtures thereof (see below).

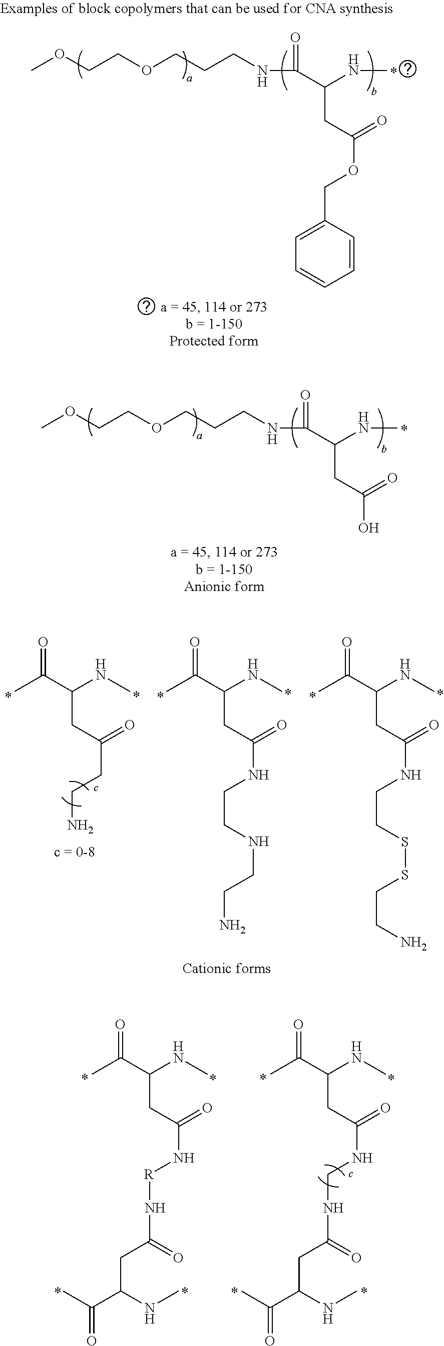

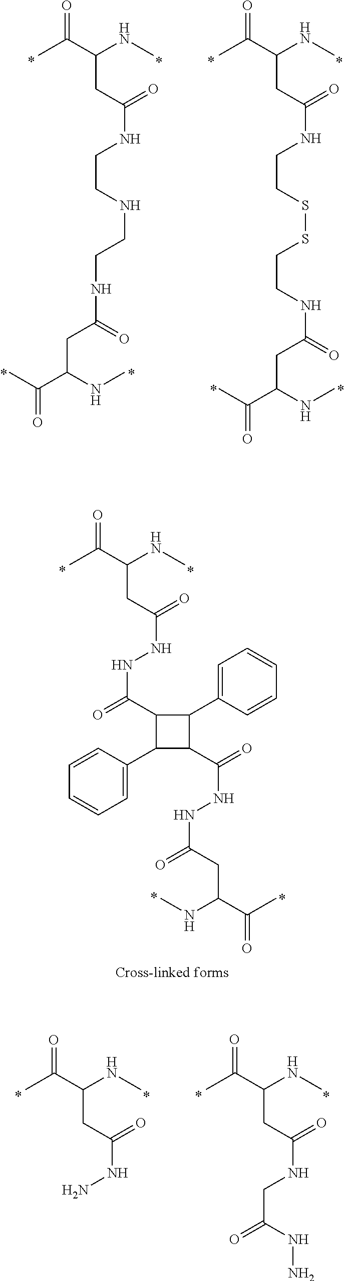

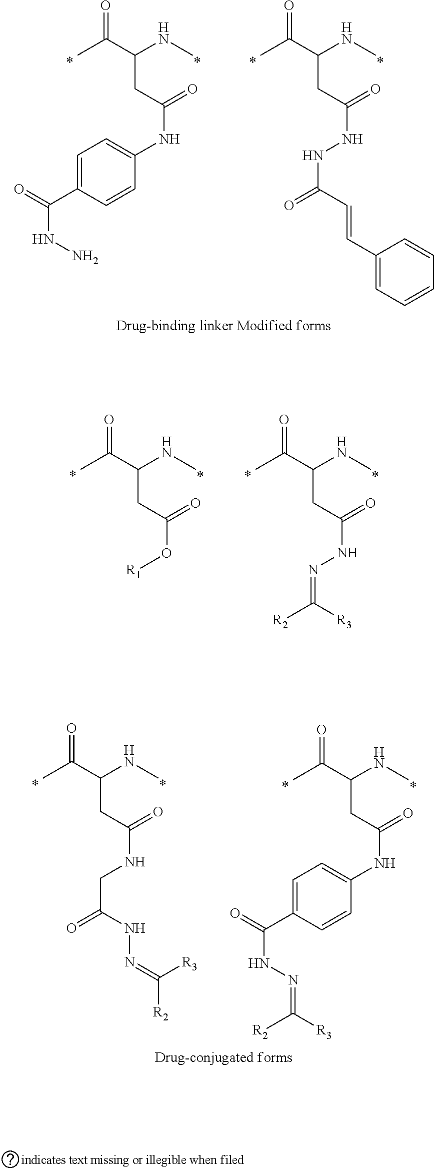

The term “diagnostic agent” refers to any biologically compatible organic, orano-metallic or inorganic compound that may be used for diagnosis. For example, diagnostic agents include imaging agents containing radioisotopes such as indium or technetium; contrasting agents containing iodine, technetium, iron oxide, gadolinium or other T1 and T2 contrast agents; enzymes such as horse radish peroxidase, GFP, alkaline phosphatase, beta-galactosidase or other therapeutic proteins; fluorescent substances such as europium derivatives or other natural and synthetic fluorophores; luminescent substances such as N-methylacrydium derivatives or the like.

In a further embodiment, the present invention relates to compositions wherein the agent is one or more of the following: a metal binding diagnostic protein imaging agent, a metal binding diagnostic enzyme imaging agent, a metal binding diagnostic fluorophore imaging agent, a metal binding diagnostic nuclear-imaging agent, a metal binding diagnostic paramagnetic imaging agent, a metal binding peptide therapeutic agent, a metal binding protein therapeutic agent, a metal binding peptide therapeutic agent, a metal binding organic compound therapeutic agent, a metal binding peptidomimetic therapeutic agent, a metal binding deoxyribonucleic acid therapeutic agent, a metal binding ribonucleic acid therapeutic agent, a metal binding oligonucleotide therapeutic agent, a metal binding nucleic acid therapeutic agent, a metal binding oligosaccharide therapeutic agent, a metal binding antibody therapeutic agent or a metal binding proteoglycan therapeutic agent. In a further embodiment, the present invention relates to the above described compositions wherein more than one type of agent forms a coordinate bond with the metal binding domain linked to a hydrophobic group or hydrophilic group.

It is understood that the term “diagnosis” as it refers to the use of the composition indicates that the composition will be used as a contrast agent to obtain an image of the patient's body to determine the region where the composition of the present invention accumulates. This information will have diagnostic value in a patient with various diseases since most diseases cause vascular changes that can be imaged or diagnosed by the compositions of the present invention. Non-limiting examples of these diseases include cancer, inflammation, arthritis, stroke, and other vascular abnormalities. In addition, compositions of the present invention can be made to localize and accumulate at site(s) where there are specific antigens, cell markers, or molecules in the body which can help in the diagnosis to determine the presence and localization of specific molecules or antigens in the body. This approach is known to those with ordinary skill in the art.

Another example of a diagnostic agent is radionuclides, which may be detected using positron emission tomography (PET) or single photon emission computed tomography (SPECT) imaging or other methods known to one of skill in the art. In one embodiment, the composition of the present invention comprises a paramagnetic contrast agent, such as gadolinium, cobalt, nickel, manganese and iron oxide. Such moieties may be chelated to their own metal binding domain which in turn could be coordinated to the metal ion found in the bridge to the block copolymer segments.

Non-limiting examples of agents include insulin, growth factors, hormones, cytokines, growth hormone (GH, somatropin), nerve growth factor (NGF), lysostaphin, GLP-1, brain-derived neurotrophic factor (BDNF), enzymes, endostatin, angiostatin, trombospondin, urokinase, interferon, blood clotting factors (VII, VIII), and any molecule able to bind metal ions.

The term “therapeutic agent” refers to biologically active agents, prodrugs, or drugs, including, for example, any organic or organometallic small molecule compound (e.g., a molecule with a molecular weight of less than about 1,000), polymeric species (including nucleic acids (DNA and RNA), proteins, peptides, hormones, carbohydrates, and derivatives thereof), lipids and mixtures thereof, wherein said drug or agent can be administered in vivo (in humans or animals) for the treatment of a disease, condition, or disorder.

Therapeutic agents include signal transduction inhibitors, drugs that may prevent the ability of cancer cells to multiply quickly and invade other tissues. One class of therapeutic agents that can be used in the nanoassembly formulations includes 90 kDa heat shock protein (HSP90) inhibitors and topoisomerase II inhibitors. HSP90 is a molecular chaperone that plays a crucial role in maintaining cell viability. HSP90 inhibitors such as geldanamycin and its analogues (e.g., 17-AAG) bind to N-terminus of HSP90 dimers. Anticancer drugs like doxorubicin target intermediate the topoisomerase II complex to induce apoptosis by intercalating into DNA.

Specific examples of therapeutic agents of the invention that can be used to form bioconjugates with the polymers described herein include, but are not limited to, aclarubicin, apicidin, 17-allylamino-17-demethoxygeldanamycin (17-AAG), cyclopamine-KAAD, cucurbitacin, docetaxel, dolastatin, doxorubicin (adriamycin), geldanamycin, fenritinide, herbimycin A, 2-methoxyestradiol (an angiogenesis inhibitor), paclitaxel, radicicol, rapamycin, triptolide, wortmannin, and the various combinations thereof. Other therapeutic agents include proteasome inhibitors such as bortezomib, and benzyloxycarbonyl-L-leucyl-L-leucyl-L-leucinal (“Z-Leu-Leu-Leu-H (aldehyde)”), which is also a potent inhibitor of Cathepsin K. See Votta et al., J. Bone Miner. Res., 12, 1396 (1997). Additional therapeutic agents that have suitably reactive carbonyl groups, or groups that can employ a linker, that can be used to form bioconjugates can be found in The Merck Index, 12^{th} Edition (1996).

Further specific examples of suitable therapeutic agents that can be linked to the polymers include aclacinomycins, 9-aminocamptothecin, aminopterin, ara-C (cytarabine), azaserine, biricodar, bleomycins, cactinomycin, calusterone, camptothecin, carboplatin, carboquone, caminomycin, carubicin, chlormadinone acetate, chromomycins, cisplatin, CPT 11, cyclophosphamide, cytarbin, cytosine arabinoside, dactinomycin, daunorubicin, 6-diazo-5-oxo-L-norleucine, dichloromethotrexate, docetaxel, doxorubicin, dromostanolone propionate, dromostanolone, emitefur, epirubicin, estramustine, etoposide, exemestane, flavopiridol, 5-fluorouracil, formestane, gemcitabine, hexamethyl melamine, idarubicin, irinotecan, leurosidine, medroxyprogesterone, megestrol acetate, melengestrol, melphalan, menogaril, 6-mercaptopurine, methopterin, methotrexate, methoxsalen, mitomycin-C, mitoxantrone, nogalamycin, onapristone, phenesterine, pipobroman, piposulfan, pirarubicin, podophyllotoxin, porfiromycin, prednimustine, rubitecan, sobuzoxane, spironolactone, streptonigrin, teniposide, tenuazonic acid, testolactone, topotecan, tretinoin, triaziquone, trimetrexate, uredepa, valrubicin, valspodar, vinblastine, vincamine, vincristine, vindesine, and zorubicin. Each of these drugs has at least one hydroxyl, amino, carboxyl, ketone, or thiol group that can form a bond with a linker of the invention for use in the nanoassemblies.

Other specific therapeutic agents that can be employed in the nanoassembly formulations, optionally by covalently bonding the agent to the polymer with a linker, include antineoplastic agents such as tipifamib, gefitinib, cetuximab, oxaliplatin, ansamitocin, arabinosyl adenine, mercaptopolylysine, busulfan, chlorambucil, mitotane, procarbazine hydrochloride, plicamycin, aminoglutethimide, estramustine phosphate sodium, flutamide, leuprolide acetate, tamoxifen citrate, trilostane, amsacrine, asparaginase, interferon, vinblastine sulfate, vincristine sulfate, carzelesin, taxotane, daunomycin; anti-inflammatory agents such as indomethacin, ibuprofen, ketoprofen, dichlofenac, piroxicam, tenoxicam, naproxen, aspirin, and acetaminophen; sex hormones such as testosterone, estrogen, progestone, estradiol; antihypertensive agents such as captopril, ramipril, terazosin, minoxidil, and parazosin; antiemetics such as ondansetron and granisetron; antibiotics such as metronidazole, and fusidic acid; cyclosporine; prostaglandins; biphenyl dimethyl dicarboxylic acid, antifungal agents such as ketoconazole, and amphotericin B; steroids such as triamcinolone acetonide, hydrocortisone, dexamethasone, prednisolone, and betamethasone; cyclosporine, and functionally equivalent analogues, derivatives, or combinations thereof.

As used herein, the drug names adriamycin and doxorubicin are used interchangeably in the context of forming a drug conjugate. The term “adriamycin” is sometimes used to specifically refer to the HCl salt of doxorubicin. Therefore, one skilled in the art would readily recognize that both doxorubicin and its HCl salt will form the same drug conjugate, in various embodiments.

The term “therapeutically effective amount” is intended to qualify the amount of a therapeutic agent required to relieve to some extent one or more of the symptoms of a disease or disorder, including, but not limited to: 1) reduction in the number of cancer cells; 2) reduction in tumor size; 3) inhibition of (i.e., slowing to some extent, preferably stopping) cancer cell infiltration into peripheral organs; 3) inhibition of (i.e., slowing to some extent, preferably stopping) tumor metastasis; 4) inhibition, to some extent, of tumor growth; 5) relieving or reducing to some extent one or more of the symptoms associated with the disorder; and/or 6) relieving or reducing the side effects associated with the administration of anticancer agents.

The terms “treat” and “treatment” refer to any process, action, application, therapy, or the like, wherein a mammal, including a human being, is subject to medical aid with the object of improving the mammal's condition, directly or indirectly.

The term “inhibition,” in the context of neoplasia, tumor growth or tumor cell growth, may be assessed by delayed appearance of primary or secondary tumors, slowed development of primary or secondary tumors, decreased occurrence of primary or secondary tumors, slowed or decreased severity of secondary effects of disease, arrested tumor growth and regression of tumors, among others. In the extreme, complete inhibition, can be referred to as prevention or chemoprevention.

The term “PEG” refers to poly(ethylene glycol) and derivatives thereof. The molecular weight of the PEG chain can be about 500 to about 20,000. In certain embodiments, the PEG group can have a molecular weight of about 2,000 to about 15,000, about 3,500 to about 12,000, or about 3,000 to about 9,000. In other embodiments, the PEG groups can have a molecular weight of about 4,000 or about 7,000. PEG derivatives include PEG groups with amine or amide groups at one or both ends, and carboxylic acid groups at one or both ends.

The term “linker”, “linkers” or “linking group” refers to a covalent bond or a chain, typically a carbon chain, for example, a C₁-C₂₀ chain, that covalently links two moieties together. The chain is optionally interrupted by one or more nitrogen atoms, oxygen atoms, carbonyl groups, (substituted)aromatic rings, or peptide bonds, and/or one of these groups may occur at one or both ends of the chain that forms the linker. Therefore, either or both ends of the linker can terminate in an oxy, amino, carboxyl, oxycarbonyl, amide, carbonate, carbamate, sulfonyl, or hydrazone group. Accordingly, the linker can also be a chain of one to about five amino acids, of the same type, such as poly L-glycine, poly L-glutamine, or poly L-lysine, or of different types of amino acids. In some embodiments, the linker can be a PEG group, with up to 20 repeating units. Examples of simple linkers include succinimidyl groups, sulfosuccinimidyl groups, maleimidyl groups, and various C₂-C₁₂ diamines and dicarboxylic acids. Many linkers are well known in the art, and can be used to link a polymer described herein to another therapeutic agent.

Linkers include amine-to-amine cross-linkers, amine-to-sulfhydryl cross-linkers, carboxyle-to-amine cross-linkers, photoreactive cross-linkers, sulfahydryl-to-carbohydrate cross-linkers, sulfhydryl-to-hydroxyl cross-linkers and sulfhydryl-to-sulfhydryl cross-linkers as described in more detail at http://www.piercenet.com/browse.cfm?fldID=203. See also for example, the linkers described by Sewald and Jakubke in Peptides: Chemistry and Biology, Wiley-VCH, Weinheim (2002), pages 212-223; and by Dorwald in Organic Synthesis on Solid Phase, Wiley-VCH, Weinheim (2002).

The phrase “pharmaceutically acceptable” refers to those compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio.

The phrase “pharmaceutically acceptable salts” refers to ionic compounds wherein a parent non-ionic compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include conventional non-toxic salts and quaternary ammonium salts of the parent compound formed, for example, from non-toxic inorganic or organic acids. Non-toxic salts can include those derived from inorganic acids such as hydrochloric, hydrobromic, hydroiodic, sulfuric, sulfamic, phosphoric, nitric and the like. Salts prepared from organic acids can include those such as acetic, 2-acetoxybenzoic, ascorbic, benzenesulfonic, behenic, benzoic, citric, ethanesulfonic, ethane disulfonic, formic, fumaric, gentisinic, glucaronic, gluconic, glutamic, glycolic, hydroxymaleic, isethionic, isonicotinic, lactic, maleic, malic, methanesulfonic, oxalic, pamoic (1,1′-methylene-bis-(2-hydroxy-3-naphthoate)), pantothenic, phenylacetic, propionic, salicylic, sulfanilic, toluenesulfonic, stearic, succinic, tartaric, bitartaric, and the like. Certain compounds can form pharmaceutically acceptable salts with various amino acids. For a review on pharmaceutically acceptable salts see Berge et al., J. Pharm. Sci. 1977, 66(1), 1-19, which is incorporated herein by reference. In certain embodiments, it may be useful to employ salts of various organic moieties on the polymers of the invention. For example, the polyamide block polymer may include one or more acidic or basic side chains that may form salts under appropriate conditions.

The pharmaceutically acceptable salts of the compounds described herein can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., (1985), 1418, the disclosure of which is incorporated herein by reference.

The phrase “low water solubility” refers to a compound that dissolves in water in an amount of less than about 200·mu·g/mL, for example, measured at neutral pH. In some embodiments, compounds that have low water solubility will dissolve at less than about 100·mu·g/mL. In other embodiments, low water solubility refers to solubility of less than about 75·mu·g/mL, less than about 50·mu·g/mL, or less than about 25·mu·g/mL. Many drugs are lipophilic, and therefore have poor water solubility, making it difficult to administer them in a safe and effective manner. Suitable water solubility is of particular importance for parenteral administration, therefore the nanoassembly formulations described herein provide a significant advantage for administering these drugs, particularly for administering drugs in combination therapy.

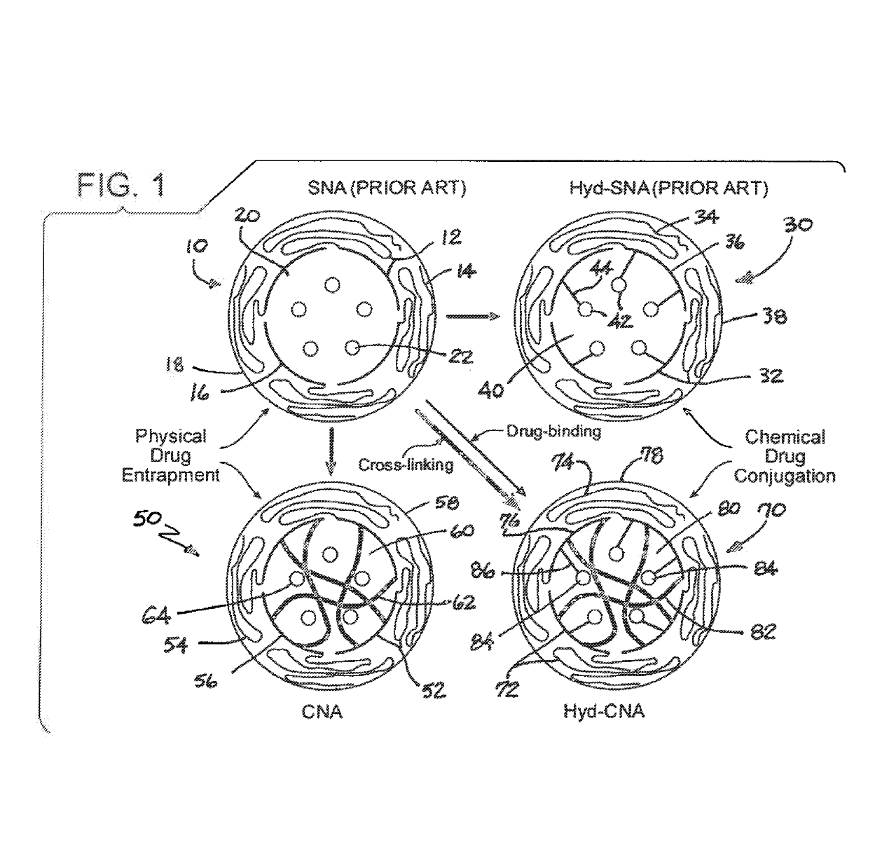

Reference is now made to FIG. 1 showing four different types of nanoassemblies. A self-assembled nanoassembly (SNA) 10 includes multiple block copolymers 12. Each block copolymer includes a hydrophilic segment 14 and a hydrophobic segment 16. The block copolymers 12 self-assemble into a biocompatible shell 18 formed from the hydrophilic segment 14 and a core 20 formed by the hydrophobic segment 16. An active agent 22 may be physically entrapped in the core 20. This type of nanoassembly 10 is known in the art.

A drug-bound drug self-assembled nanoassembly (Hyd-SNA) 30 includes a plurality of block copolymers 32. Each block copolymer includes a hydrophilic segment 34 and a hydrophobic segment 36. The block copolymers 32 self-assemble into a biocompatible shell 38 formed by the hydrophilic segments 34 and a core 40 formed by the hydrophobic segments 36. An active agent 42 is maintained in the core 40 and bound to the block copolymers 32 by drug-binding linkers 44. This type of nanoassembly 30 is also known in the art.

A cross-linked nanoassembly (CNA) 50 comprises a plurality of block copolymers 52. Each block copolymer includes a hydrophilic segment 54 and a hydrophobic segment 56. The CNA 50 includes a biocompatible shell 58 formed from the hydrophilic segments 54 surrounding an interior core 60 formed from the hydrophobic segments 56. Block copolymer cross-linkers 62 link the block copolymers 52 together by binding between the hydrophilic segments 54. An active agent 64 is physically entrapped in the core 60 of the CNA 50.

The fourth type of nanoassembly is the active agent bound and cross-linked nanoassembly (Hyd-CNA) 70. The Hyd-CNA 70 comprises a plurality of block copolymers 72. Each block copolymer 72 includes a hydrophilic segment 74 and a hydrophobic segment 76. Block copolymers 72 are assembled into a nanoassembly including a biocompatible shell 78 formed by the hydrophilic segment 74 and an interior core 80 surrounded by the hydrophobic segment 76. Block copolymer cross-linkers 82 link the hydrophobic segments 76 of the block copolymers 72. An active agent 84 is maintained in the core 80. As illustrated the active agent 84 is bound by drug-binding linkers 86 to the hydrophobic segment 76 of the block copolymers 72.

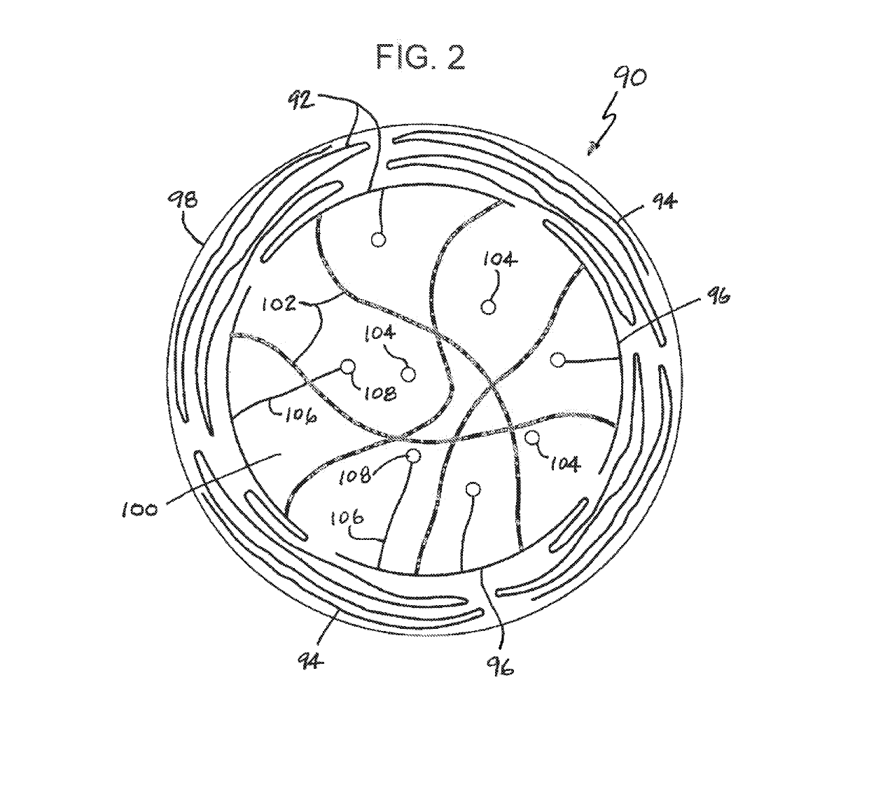

Reference is now made to FIG. 2 illustrating yet another embodiment of a nanoassembly 90. This embodiment comprises a plurality of block copolymers 92. Each block copolymer includes a hydrophilic segment 94 and a hydrophobic segment 96. The block copolymers are assembled to provide a biocompatible shell 98, made from the hydrophilic segments 94, and an interior core 100 contained within the hydrophobic segments 96. Block copolymer cross-linkers 102 bind or connect the hydrophobic segments 94 of the block copolymers 92 together. A first active agent 104 is physically entrapped in the core 100. Drug-binding linkers 106 covalently bind a second active agent 108 which is also held in the core 100.

The novel nanoassemblies 50, 70 and 90 disclosed herein, have a diameter of about 5 nm to about 200 nm. The nanoassemblies 50, 70 and 90 further have a cross-linking yield of about 1% to about 50% and a drug loading of about 1% to about 60% by weight. The higher the cross-linking yield, the stronger the block copolymers 52, 72, 92 are bound together. This produces nanoassemblies of enhanced stability and narrower size range. Cross-linking yields are easily controlled by adjusting the molar ratio of cooperating linking functional groups on the block copolymers and the cross-linkers as well as by modulating the feeding ratio of cross-linker as compared to block copolymer.

The drug-binding linkers 86, 106 and the cross-linkers 62, 82, 102 include both permanent linkers and degradable linkers. The drug-binding linkers 86, 106 and cross-linkers 62, 82, 102 may be selected from a group consisting of (1) an aliphatic compound with amino, carboxyl, hydroxyl, ketone or thiol groups and cross-linking formed through amide, ester, carbamate, imine, hydrazone, and disulfide bonds (2) an aromatic compound with amino, carboxyl, hydroxyl, ketone or thiol groups and cross-linking formed through amide, ester, carbamate, imine, hydrazone, and disulfide bonds and (3) mixtures thereof.

More specifically, block copolymer cross-linkers 62, 82, 102 include functional groups capable of binding and linking with cooperating functional groups on the block copolymers, not the active agents. In contrast drug-binding linkers 86, 106 include functional groups capable of binding and linking with cooperating functional groups on an active agent, not the block copolymer.

Where the first active agent 104 is physically entrapped in the core of the nanoassembly and the second active agent 108 is covalently bound to the block copolymer 92, the drug-binding linkers 106 include a functional group that (a) will bind and link with a functional group on the second active agent 108 and (b) will not bind and link with a functional group on the first active agent. Thus, for example, an active agent 108 with a ketone group will be chemically conjugated and bound to a block copolymer 92 including a hydrazide drug-binding linker 106 while the other active agents 104, not including a ketone group, will simply be physically entrapped in the core.

Similarly, a variety of combinations among functional groups may be used to control chemical and physical drug entrapment. For example, two different active agents may be covalently bound to the block copolymer in a desired ratio by using (a) a first quantity of a first drug-binding linker having a first functional group that will selectively bind and link with a functional group of the first active agent and (b) a second quantity of a second drug-binding linker having a second functional group that will selectively bind and link with a functional group of the second active agent.

Where it is desired to both covalently bind and physically entrap the same active agent in a nanoassembly in order to lengthen the time over which the active agent is released from the nanoassembly, one provides a molar ratio of drug-binding linker that is sufficient to covalently bind the desired amount of active agent, the remaining active agent being subject to physical entrapment.

Unlike permanent linkers, degradable linkers include functional groups that degrade when subjected to certain stimuli or conditions. The degradable cross-linkers 62, 82, 102 and drug-binding linkers 86, 106 may include pH degradable bonds, light degradable bonds, heat degradable bonds and/or bonds degradable by enzymatic activity.

Linkers 62, 82, 86, 102, 106 with pH degradable bonds include, but are not limited to hydrazone and citraconyl double bonds.

Linkers 62, 82, 86, 102, 106 with light degradable bonds include but are not limited to o-nitrobenzyl ester, o-nitrobenzyl ether, benzoin, phenacyl ester, and coumarin derivatized linkers.

Linkers 62, 82, 86, 102, 106 with heat degradable bonds include but are not limited to those with functional groups that are cleaved by hydrolysis such as an ester.

Linkers 62, 82, 86, 102, 106 with bonds degradable by enzymatic activity include but are not limited to those with functional groups that are cleaved by protease such as an amide or an ester.

Active agents 64, 84, 104, 108 may be selected from a group consisting of a diagnostic agent, an imaging agent, a therapeutic agent, multiple diagnostic agents, multiple imaging agents, multiple therapeutic agents and mixtures thereof. The first active agent 104 and second active agent 108 may be the same diagnostic agent, imaging agent, therapeutic agent, multiple diagnostic agents, multiple imaging agents, multiple therapeutic agents and mixtures thereof or different diagnostic agents, imaging agents, therapeutic agents, multiple diagnostic agents, multiple imaging agents, multiple therapeutic agents and mixtures thereof.

At least one of the active agents or guest molecules 64, 84, 104, 108 is selected from a group consisting of a small molecule having less than 1,000 atoms, a large molecule having at least 1,000 atoms, a peptide, a plasmid DNA, siRNA, a fluorescent dye, a contrast agent and mixtures thereof.

In one possible embodiment the first active agent and second active agent include at least one hydrophobic agent and at least one hydrophilic agent in a single nanoassembly. For example, a nanoassembly may be prepared including a first, hydrophobic active agent (e.g. acridine yellow, nile red, pyrene, geldanamycin, 17-AAG, SN38, docetaxel, 3PO, 4IPP, wortmannin) covalently bound to the block copolymers physically entrapped in the core and a second hydrophilic active agent (e.g. doxorubicin-HCl, methramycin analogues, Alexa, FITC, IR280), physically entrapped in the core.

In accordance with yet another aspect, a method is provided for making a biocompatible nanoassembly 90. The method comprises cross-linking block copolymers 92 including drug-binding linkers 106 and cross-linkers 102, covalently binding a second active agent 108 to the block copolymers 92 and physically entrapping a first active agent 104 in the core of the nanoassembly protected by the biocompatible shell formed by the cross-linked block copolymers. The method further includes providing a plurality of block copolymers 92 with drug-binding linkers 106 and block copolymer cross-linkers 102.

In accordance with one particularly simple and effective way for making biocompatible nanoassemblies 90 the method includes dissolving (a) block copolymers 92, including both drug-binding linkers 106 and block copolymer cross-linkers 102, (b) a first active agent 104 and (c) a second active agent 108 together in a solvent and simultaneously completing the cross-linking, covalent binding and physical entrapping steps. Further the method includes simultaneously completing the cross-linking, covalent binding and physical entrapping steps while providing a cross-linking yield between 1% and 50%, a drug loading of between 1% and 60% by weight and maintaining a nanoassembly having a diameter of 5 to 200 nm.

In prior art approaches such as disclosed in U.S. Pat. No. 7,332,527 to Bronich et al. and U.S. Pat. No. 6,383,500 to Wooley et al., polymer micelles must first be molecularly complexed or self-assembled prior to cross-linking. This multistep process is relatively time consuming and usually requires purification steps that limit yields. In contrast, in the present method the block copolymers are assembled and cross-linked simultaneously and there are no separate purification steps required. Thus assembly formation stops once the cross-linked nanoassemblies reach the most thermodynamically stable condition. Advantageously this results in highly reproducible particle size, improved product yields, concurrent payload entrapment and cross-linking, and consistent particle properties after lyophilization.

Solvents useful in the present method include but are not limited to deionized water, buffer solutions, and organic and aqueous mixed solutions.

Nanoassemblies 90 prepared by the present method can be suitably formulated into pharmaceutical compositions for administration to mammalian/human subjects in a biologically compatible form suitable for administration in vivo. Accordingly, in certain embodiments, a pharmaceutical composition is provided that includes nanoassemblies as described herein in a mixture with a suitable diluent or carrier. Suitable diluents or carriers include saline. Such formulations may be prepared so that they are isotonic with mammalian/human fluids, such as blood or various tissue environments. In certain embodiments, it may also be desirable to prepare hypertonic or hypotonic preparations. In other embodiments, the compositions can be prepared and used for in vitro experimentation, for example, in various strains in diagnostic procedures.

The compositions containing nanoassemblies 90 can be prepared by known methods for the preparation of pharmaceutically acceptable compositions that can be administered to subjects, such that an effective quantity of the therapeutic agent within the nanoassemblies 90 is combined in a mixture with a pharmaceutically acceptable vehicle. Suitable vehicles are described, for example, in Remington's Pharmaceutical Sciences (2003, 20^{th} Ed.), in The United States Pharmacopeia: The National Formulary (USP 24 NF19) published in 1999, and in the Handbook of Pharmaceutical Additives (compiled by Michael and Irene Ash, Gower Publishing Limited, Aldershot, England (1995)). On this basis, the compositions include, albeit not exclusively, solutions of the nanoassemblies 90 in association with one or more pharmaceutically acceptable vehicles or diluents, and contained in buffered solutions with a suitable pH and iso-osmotic with the physiological s. In this regard, reference can be made to U.S. Pat. No. 5,843,456 (Paoletti et al.). In one embodiment, the pharmaceutical compositions can be used to enhance biodistribution and drug delivery of therapeutic agents in a therapeutically effective amount, such as a drug linked to a polymer of the nanoassembly.

The nanoassemblies 90 described herein can be administered to a subject in a variety of forms depending on the route of administration selected, as is readily understood by those of skill in the art. The nanoassemblies 90 can be administered, for example, by oral, parenteral, buccal, sublingual, nasal, rectal, patch or pump administration and the pharmaceutical compositions formulated accordingly. Parenteral administration includes intravenous, intraperitoneal, subcutaneous, intramuscular, intrasternal, transepithelial, nasal, intrapulmonary, intrathecal, rectal and infusion modes of administration. Parenteral administration may be by continuous infusion over a selected period of time.

Injectable preparations, for example, sterile injectable aqueous or oleaginous suspensions can be formulated according to the known art using suitable dispersing or wetting agents and suspending agents. The sterile injectable preparation can also be a sterile injectable solution or suspension in a nontoxic parenterally acceptable diluent or solvent. Among the acceptable vehicles and solvents that can be employed are water, Ringer's solution, and isotonic sodium chloride solution. In addition, sterile, solutions of a nanoassembly can be prepared in water. Under ordinary conditions of storage and use, these preparations may contain a preservative, for example, to prevent the growth of microorganisms.

The pharmaceutical forms suitable for injectable use include sterile aqueous solutions for the extemporaneous preparation of sterile injectable solutions. The formulation should be sterile and should be fluid to the extent that the solution can be administered via syringe.

Compositions for nasal administration may conveniently be formulated as aerosols, drops, gels and powders. Aerosol formulations typically comprise a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device such as a single dose nasal inhaler or an aerosol dispenser fitted with a metering valve which is intended for disposal after use. Where the dosage form comprises an aerosol dispenser, it will contain a propellant which can be a compressed gas such as compressed air or an organic propellant such as fluorochlorohydrocarbon. The aerosol dosage forms can also take the form of a pump-atomizer.

The compositions described herein can be administered to an animal alone or in combination with conventional therapeutic formulations including pharmaceutically acceptable carriers the proportion of which is determined by the solubility and chemical nature of the compound, chosen route of administration and standard pharmaceutical practice. In an embodiment, the pharmaceutical compositions are administered in a convenient manner such as by direct application to the infected site, e.g. by injection (subcutaneous, intravenous, parenteral, etc.). In case of respiratory infections, it may be desirable to administer the nanoassemblies 90 of the invention and compositions comprising same, through known techniques in the art, for example by inhalation. Depending on the route of administration (e.g. injection, oral, or inhalation, etc.), the pharmaceutical compositions or nanoassemblies 90 or biologically active agents in the nanoassemblies 90 of the invention may be coated in a material to protect the nanoassemblies 90 or agents from the action of enzymes, acids, and other natural conditions that may inactivate certain properties of the composition or its encapsulated agent.

In addition to pharmaceutical compositions, compositions for non-pharmaceutical purposes are also included within the scope of the invention. Such non-pharmaceutical purposes may include the preparation of cosmetic formulations, or for the preparation of diagnostic or research tools. In one embodiment, the therapeutic agents or nanoassemblies 90 comprising such agents can be labeled with labels known in the art, such as florescent or radio-labels, or the like. In some embodiments, one or more of the drugs of the polymer can be replaces with a diagnostic agent.

The invention also provides a delivery system that can be used to deliver biologically active agents or formulations or pharmaceutical compositions. In one embodiment, the invention includes the delivery of a combination of cancer therapeutic agents in therapeutically effective amounts.

Another aspect of the invention includes a method of delivering biologically active agents to treat a disease, condition, or disorder in a subject in need thereof comprising administering a therapeutically effective amount of an agent-loaded nanoassembly to a subject. In one embodiment, the disease, condition or disorder is cancer or drug resistant cancers, infectious disease or an autoimmune disease.

The dosage of the nanoassemblies 90 can vary depending on many factors such as the pharmacodynamic properties of the nanoassembly, the biologically active agent, the rate of release of the agent from the nanoassemblies 90, the mode of administration, the age, health and weight of the recipient, the nature and extent of the symptoms, the frequency of the treatment and the type of concurrent treatment, if any, and the clearance rate of the agent and/or nanoassembly in the subject to be treated.

For example, in some embodiments, a dose of a nanoassembly formulation equivalent to about 1 mg mL⁻¹ to about 100 mg mL⁻¹ can be administered to a patient. In certain other embodiments, the nanoassembly formulation includes about 2-20, about 5-15, or about 10 mg mL⁻¹. The specific doses of the compounds administered according to this invention to obtain therapeutic and/or prophylactic effects will, of course, be determined by the particular circumstances surrounding the case, including, for example, the compounds administered, the route of administration, the condition being treated and the individual being treated. A typical daily dose (administered in single or in divided doses) can contain a dosage level of from about 0.01 mg/kg to about 150 mg/kg of body weight of an active therapeutic agent described herein. In some embodiments, about 5-10, about 10-20, about 20-40, about 25-50, about 50-75, about 75-100, or about 100-150 150 mg/kg of body weight of a therapeutic agent are provided in a dose. In other embodiments, about 5, 10, 15, 20, 25, 30, 40, 50, 60, 70, 75, 80, 90, 100, 110, 120, 125, 140, or 150 mg/kg of body weight of a therapeutic agent are delivered in a dose. Often times, daily doses generally will be from about 0.05 mg/kg to about 20 mg/kg and ideally from about 0.1 mg/kg to about 10 mg/kg.

One of skilled in the art can determine the appropriate dosage based on the above factors. The nanoassemblies 90 may be administered initially in a suitable dosage that may be adjusted as required, depending on the clinical response. For ex vivo treatment of cells over a short period, for example for 30 minutes to 1 hour or longer, higher doses of nanoassemblies 90 may be used than for long term in vivo therapy.

The nanoassemblies 90 can be used alone or in combination with other agents that treat the same and/or another condition, disease or disorder. In another embodiment, where either or both the nanoassembly or biologically active agent is labeled, one can conduct in vivo or in vitro studies for determining optimal dose ranges, drug loading concentrations and size of nanoassemblies 90 and targeted drug delivery for a variety of diseases.

The nanoassemblies can be used for modular applications in which multiple individual nanoassemblies can be replaced without affecting their properties to entrap multiple active agents, control release profiles, and achieve differential tissue/cell targeting in controlled manners.

The following synthesis and examples are presented to further illustrate the various nanoassembly embodiments but they are not to be considered as limited thereto.

### Example 1

### Introduction

Drug release rates are generally controlled by drug molecule diffusion and drug carrier erosion. Nanoscale drug carriers have relatively small volume to entrap drug payloads in comparison to larger drug delivery platforms (e.g. film, patches, matrices, and micro particles), leading to the development of various drug carriers that have degradable core, shell, or drug-binding linkers. Although there are many bottom-up and top-down approaches to engineer the properties of nanomaterials, one of the widely used methods to prepare nanoscale drug carriers is to exploit the self-assembling phenomenon between amphiphilic block polymers. For example, block copolymers that possess hydrophilic and hydrophobic segments form micellar nanoassemblies, referred to as polymer micelles, providing a hydrophobic nano-compartment enveloped with a hydrophilic protection shield in aqueous solutions. The polymer micelles have been used in several clinical applications for the delivery of various biomedical materials, including anticancer drugs, therapeutic proteins, plasmid DNA, siRNA, contrast agents, and fluorescence probes. Nevertheless, polymer micelles and other supramolecular assemblies (e.g. self-assembled vesicles, lamellar, or layers) have innate limitations of particle stability in diluted conditions as they dissociate below critical micelle concentrations or the Krafft temperature. Despite these limitations, self-assemblies still have pharmaceutical advantages mainly due to their easy preparation method to design formulations for various therapeutic agents. To improve stability, molecular self-assemblies are often cross-linked in the core and shell. However, pharmaceutical differences between self-assembled and cross-linked nanoassemblies are not fully understood.

We have studied functional polymer micelles from self-assembling block copolymers, which can fine-tune drug release patterns. Our results demonstrate that both fast and slow drug release rates can be equally effective to suppress cancer cell growth, suggesting that drug release rates and total drug payload concentrations cannot be simply used to predict cytotoxicity of drug carriers. We hypothesized that drug carriers will achieve the maximum cytotoxicity by synchronizing drug release and cell growth rates, potentially providing a better measure to determine optimal drug release rates. This hypothesis is formulated based on the fact that most anticancer drugs are designed to kill fast-growing cells, and that drug carriers with varied drug release rates do not always show the same cytotoxicity in vitro and therapeutic efficacy in vivo. Results from previous literatures also support this hypothesis, addressing that low-dose metronomic chemotherapy appeared highly effective in vivo, and that cell cycle control would be a promising strategy to maximize drug efficacy to kill cancer cells. Although these facts emphasize the pharmaceutical importance of drug release rates on therapeutic efficacy of drug carriers, most existing drug carriers are developed mainly by focusing to increase total drug concentrations in tumors and inside cells, while the effects of drug release rates on cytotoxicity remains unclear as drug carriers can interact directly with cells and alter cell viability. For example, some polymer surfactants destabilize the cellular membrane, either increasing intracellular drug concentrations or breaking down the cells to death. Therefore, drug carriers should be non-toxic to investigate the effect of drug release rate on cytotoxicity more accurately.

For these reasons, we selected biocompatible poly(ethylene glycol)-poly(aspartate) [PEG-p(Asp)] block copolymers, which we have been using in previous studies and confirmed safety in vitro and in vivo. PEG is a biocompatible polymer used widely in biomedical applications for surface coating to avoid agglomeration, protein adsorption, and the immune response of materials in vivo, and drug carriers modified with PEG have shown to enhance blood circulation time and reduce toxicity and immune response. In addition to biocompatibility, particle size and shape were previously identified as factors that change intracellular uptake and in vivo distribution of drug carriers, indicating that 30 to 150 nm particles can accumulate in tumor tissues most effectively. As for a drug payload, an anticancer drug doxorubicin (DOX) was used in this example because DOX, alone or in combination with other drugs, is frequently used for the treatment of various cancers in clinic. In addition, DOX has strong UV-Visible absorption and fluorescence, which are beneficial for easy drug quantification. We previously confirmed that DOX is a model anticancer drug suitable for drug delivery studies because it remains chemically and biologically active during the processes of its entrapment to and release from drug carriers.

To test our hypothesis and rationally design a more effective drug carrier, we prepared four types of nanoassemblies as model nanoparticle drug carriers with similar biocompatibility (surface-modified with PEG), particle size (<100 nm), and drug entrapment yields (>10 weight %). These nanoassemblies were designed to control drug release rates by using drug-binding and cross-linkers in combination. As illustrated in FIG. 1, the first type of a nanoassembly is a block copolymer self-assembled nanoassembly, dubbed SNA, which is a polymer micelle prepared from PEG-p(Asp) block copolymers in the presence of Ca²⁺ ions as well as DOX. The second nanoassembly, Hyd-SNA, is SNA to which DOX was conjugated through a hydrazide drug-binding linker, which we previously showed to form an acid-labile hydrazone bonding with DOX. The third nanoassembly is a cross-linked nanoassembly (CNA), which is SNA cross-linked in the core with amide bonds. Lastly, the fourth nanoassembly, Hyd-CNA, is a nanoassembly newly prepared for this study by crosslinking Hyd-SNAs and incorporating hydrazide drug-binding linkers in the core in combination. DOX was entrapped through either physical entrapment to SNA and CNA, or covalent chemical conjugation to Hyd-SNA and Hyd-CNA, respectively. These four types of nanoassemblies were characterized as detailed below to study their physicochemical and biological properties in vitro. Results from this study, therefore, provide a better understanding of pharmaceutical differences between self-assembled and cross-linked nano assemblies.

### Materials and Methods

### Chemicals and Cell Line

L-Aspartic acid β-benzyl ester (BLA), triphosgene, anhydrous hydrazine, N,N′diisopropylcarbodiimide (DIC), N-hydroxysuccinimide (NHS), adipic acid, triethylamine, dimethylsulfoxide-d6 (DMSO-d6), anhydrous tetrahydrofuran (THF), anhydrous hexane, anhydrous dimethylsulfoxide (DMSO), anhydrous ethyl ether, benzene, acetate buffer solutions (pH 5.0, 10 mM), phosphate buffer solutions (pH 7.4, 10 mM), and doxorubicin hydrochloride (DOX) were purchased from Sigma-Aldrich (USA). α-Methoxy-ω-amino poly(ethylene glycol) (PEG or mPEG-NH₂) with 5 kDa molecular weight (MW) was purchased from NOF Corporation (Japan). Calcium chloride, regenerated cellulose dialysis bags with molecular weight cut off (MWCO) 6˜8 kDa and 50 kDa, Slide-A-Lyzer® G2 dialysis cassettes with MWCO 10 kDa, and Sephadex LH-20 gels were purchased from Fisher Scientific (USA). Amicon-Ultra centrifugal ultrafiltration devices with MWCO 100 kDa were purchased from Millipore (USA). A human non-small cell lung cancer A549 cell line and F12K cell culture medium were obtained from ATCC (USA). Fetal bovine serum (FBS) and phosphate buffered saline (PBS) were provided by Atlanta Biologicals (USA).

### Block Copolymer Synthesis

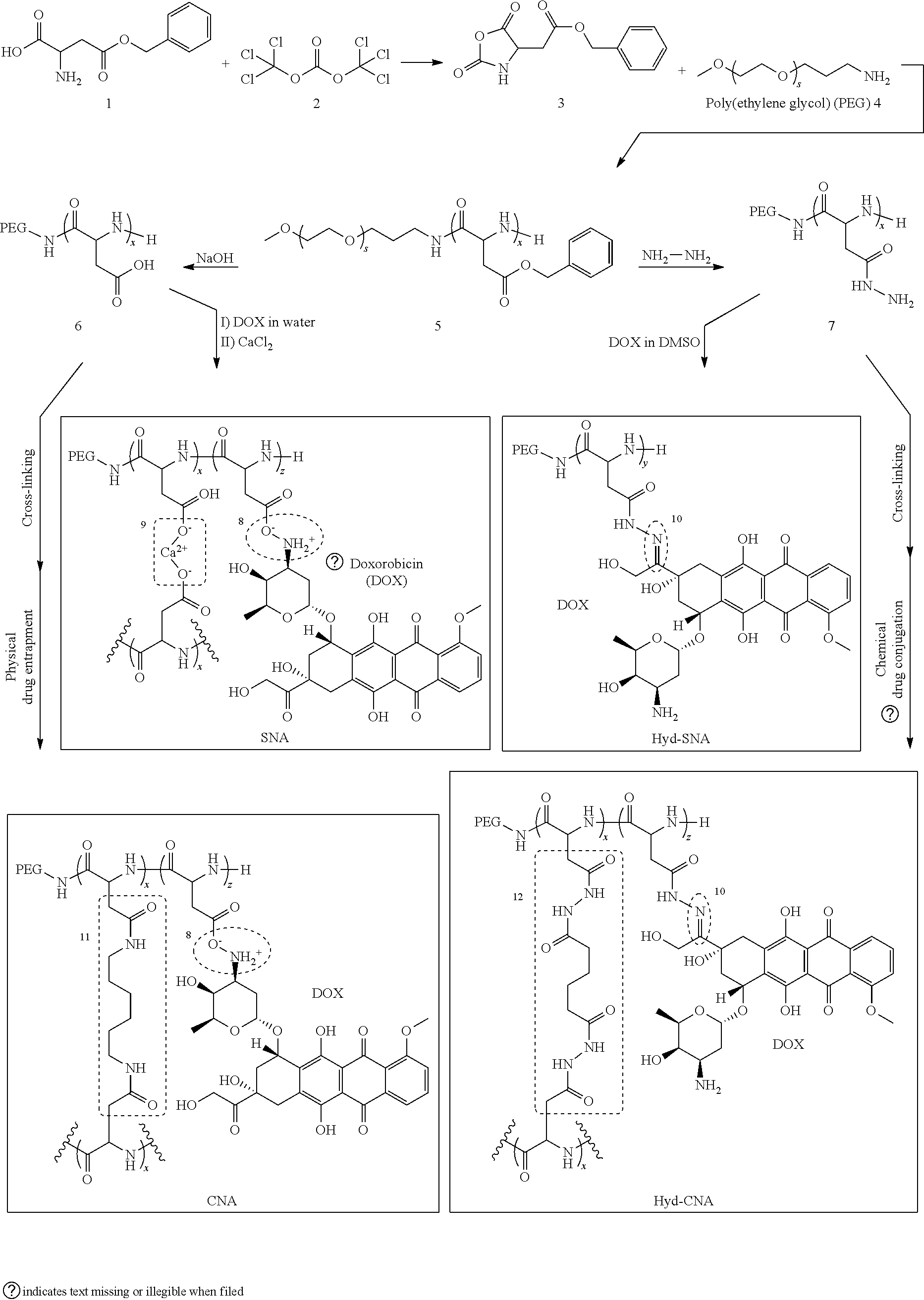

β-Benzyl-L-aspartate N-carboxy anhydride (BLA-NCA, 3) was synthesized as a monomer by the Fuchs-Farthing method. Briefly, BLA, 1, was reacted with 1.3 fold triphosgene, 2, in dry THF under nitrogen atmosphere at 45° C., 100 mg/mL, until the solution became clear. Anhydrous hexane was slowly added to the solution until NCA crystals appeared and disappeared quickly. The solution was recrystallized at −20° C. overnight. Needle-like BLA-NCA crystals were washed with anhydrous hexane and dried under vacuum for the block copolymer synthesis.

Ring-opening polymerization of BLA-NCA was conducted by using mPEG-NH₂, 4, as a macroinitiator to prepare poly(ethylene glycol)-poly(β-benzyl L-aspartate) (PEG-PBLA, 5) block copolymers. The polymerization reaction was carried out in anhydrous DMSO at 45° C. for 2 days. PEG-PBLA was precipitated in anhydrous ethyl ether, and freeze-dried from benzene.

PEG-p(Asp), 6, was prepared by deprotecting benzyl groups from PEG-PBLA in 0.1 N NaOH solution. PEG-p(Asp) in a deionized form was also prepared by removing Na salts through the dialysis of the polymer in 0.1 M HCl solution and subsequently deionized water. PEG-p(Asp) block copolymers in sodium salt and free carboxylate forms were collected by freeze drying.

The benzyl esters of PEG-PBLA block copolymers were replaced with hydrazide (Hyd) through aminolysis reaction to obtain PEG-p(Asp-Hyd), 7, block copolymers as reported previously. Excess hydrazine, 10 fold with respect to the number of BLA repeating units, was reacted with PEG-PBLA in DMSO at 40° C., 50˜100 mg/mL, for 1 h. Polymer products were purified through repetitive ether precipitation, dialysis against deionized water (MWCO 6˜8 kDa), and collected by freeze-drying.

### Preparation of Particles Entrapping DOX

We synthesized four types of DOX-loaded particles in this study. The particle structures are illustrated in FIG. 1. The synthesis steps are shown below.

SNA, 8, was synthesized by mixing sodium salt PEG-p(Asp) block copolymers with DOX in deionized water. The polymer concentration was 10 mg/mL, while DOX was added at 1:1 molar ratio with respect to carboxyl groups of PEG-p(Asp). Calcium chloride, 9, was used as a salt bridge between the block copolymer and DOX to stabilize the drug-loaded nanoassemblies. SNA was dialyzed against deionized water using MWCO 50 kDa, followed by ultrafiltration with MWCO 100 kDa to remove DOX bound weakly on the particle. SNA was freeze-dried, reconstituted in deionized water, and filtered through 0.22 μm filters prior to determining its particle size and drug loading.

Hyd-SNA, 10, was synthesized by two steps. First step was to conjugate DOX, desalted with triethylamine, to the hydrazide groups of PEG-p(Asp-Hyd) in DMSO with a 1:1 molar ratio at 30° C., 50 mg/mL, for 2 days, while the drug-conjugated polymers were purified by ether precipitation and Sephadex LH20 gel separation, followed by freeze drying. In next step, the drug-polymer conjugates were dissolved in DMSO and titrated in deionized water to form micelles. The micelles were dialyzed using a MWCO 50 kDa membrane, purified further by ultrafiltration (MWCO 100 kDa), and freeze-dried, producing Hyd-SNA. The final product was reconstituted in water and sterilized by 0.22 μm filtration.

CNA, 11, was prepared by cross-linking PEG-p(Asp) through an amide coupling reaction. Carboxyl groups of PEG-p(Asp) were reacted with 1,8-diaminooctane at 1:1 molar ratio in the presence of DIC, NHS, and DMAP (2:2:0.2 fold) in DMSO at room temperature for 3 days. The cross-linked PEG-p(Asp) was dialyzed against DMSO and then water with MWCO 50 kDa, followed by ultrafiltration (MWCO 100 kDa) and lyophilization to collect empty CNA powder. DOX and empty CNA were mixed at an equivalent molar ratio in deionized water, and purified following the steps used for SNA, 8. No calcium ions were used for CNA.

Hyd-CNA, 12, was prepared by cross-linking PEG-p(Asp-Hyd) through amidation reaction. Adipic acid was used as a cross-linker between PEG-p(Asp-Hyd) block copolymers. The cross-linking reaction was conducted in DMSO at room temperature for 3 days by adjusting the molar ratio adipic acid and the hydrazide groups of PEG-p(Asp-Hyd) to fine-tune cross-linking yields, while DIC, NHS, and DMAP were used as coupling reagent as used for aforementioned CNA synthesis. Cross-linked PEG-p(Asp-Hyd) block copolymers were purified by dialysis against DMSO and ether precipitation, and subsequently mixed with desalted DOX in DMSO at 50 mg/mL for drug conjugation at 30° C. for 2 days. DOX was entrapped in Hyd-CNA as similar to Hyd-SNA, 10. The final product, Hyd-CNA, was filter-sterilized (0.22 μm), following precipitation in ethyl ether, gel separation, and freeze drying.

### Material Characterization

Proton nuclear magnetic resonance (¹H-NMR, Varian, 400 MHz) measurements were used to determine the compositions of block copolymers and nanoassemblies in DMSO-d6 and D₂O, respectively. The number average molecular weights (Mn) of block copolymers and nanoassemblies were calculated by gel permeation chromatography (GPC, Shimadzu LC20, Japan) equipped with RI and UV detectors, using PEG standard and a phosphate buffered saline (PBS, 1×) mobile phase. Particle sizes of nanoassemblies were determined by dynamic light scattering (DLS, Zetasizer Nano 90, Malvern, UK) measurements at a 173° fixed angle. All samples were prepared in aqueous solutions at 2 mg/mL and filtered through 0.22 μm filters prior to GPC and DLS analyses. DOX loading and release were quantified using ultraviolet-visible spectrophotometry spectroscopy (UV-Vis, absorbance at 480 nm, SpectraMax M5, Molecular Devices, USA).

### Drug Release Experiment

All particles entrapping DOX were dissolved in deionized water (0.5 mg/mL), and transferred to six dialysis cassettes (MWCO 10 kDa). A group of three dialysis cassettes was used for each sample, and placed in an either pH 5.0 or pH 7.4 buffer solution (20 mM). Drug release patterns were monitored under the sink condition as the volume of dialysis medium was maintained 3,000 times greater (5 L) than the volume of the sample solution in the dialysis cassettes (1.65 mL/cassette, n=3). Sample solutions were dialyzed at 37° C. for 48 h. DOX remaining in each dialysis cassette was measured at 0, 1, 3, 6, 10, 24, 34, and 48 h. Data were converted to DOX released with respect to the initial DOX amount.

### Cytotoxicity Assay

A human non-small cell lung cancer A549 cell line was used to determine the cytotoxicity of empty nanoassemblies, DOX-loaded nanoassemblies, and free DOX. Cells were seeded in a 96-well plate (5,000 cells/well) in 100 μL F12K cell culture medium containing 10% FBS. After 24 hours, serial dilutions of the samples were added to the cell plate. Cells were then incubated at 37° C., 5% CO₂, for 72 hours, followed by cell viability measurement using a resazurin assay that indicates mitochondrial metabolic activity in live cells. Resazurin solution in PBS (10 μL, 1 mM) was added to the sample-treated cells at the end of the 72-hour treatment period. Three hours later, the fluorescence of resorufin, metabolited resazurin, was measured at 560 nm (excitation)/590 nm (emission). The half maximal inhibitory concentration (IC50) was calculated with Prism software (GraphPad, USA).

### Statistics

Data are expressed means±standard deviation (SD) from triplicate experiments unless mentioned otherwise. Statistical differences were determined by one-way analysis of variance (ANOVA) analysis. A difference was considered statistically significant when p<0.05, and denoted by an asterisk symbol (*).

### Results

### Polymer Synthesis

PEG-p(Asp) and PEG-p(Asp-Hyd) block copolymers were obtained from PEG-PBLA as previously reported. ¹H-NMR determined that PEG-PBLA block copolymer consisted of a PEG chain (Mn=5,000) and 33 aspartate repeating units, which was determined by integrating PEG (3.5 ppm) and benzyl (7.3 ppm) peaks. The block copolymer composition is denoted as 5-33, where the two numbers indicate the molecular weight of PEG×10⁻³ and the number of repeating aspartate units, respectively. PEG-p(Asp) showed a single peak with a narrow distribution (polydispersity index (PDI)<1.2), which was eluted before 5 kDa PEG used as an initiator, indicating successful polymer synthesis and absence of homopolymers. PEG-p(Asp-Hyd) also showed a narrow molecular weight distribution. Therefore, PEG-PBLA, PEG-p(Asp), and PEG-p(Asp-Hyd) block copolymers showed purity as we confirmed previously.

### Nanoparticle Preparation

SNA, Hyd-SNA, CNA, and Hyd-CNA nanoassemblies (FIG. 1) were characterized first by GPC, which showed a single peak with molecular weight of 140 to 160 kDa for CNA and Hyd-CNA and 200 to 250 kDa for SNA and Hyd-SNA. These molecular weights are relative values as calculated from a calibration curve prepared using PEG standard and a GPC column with a size exclusion limit of 3×10⁶ Da. Although the absolute molecular weight for each particle could not be determined, a single GPC peak indicates that nanoassemblies are homogeneous (data not shown). In particular, CNA and Hyd-CNA showed extremely narrow PDI (<1.15).

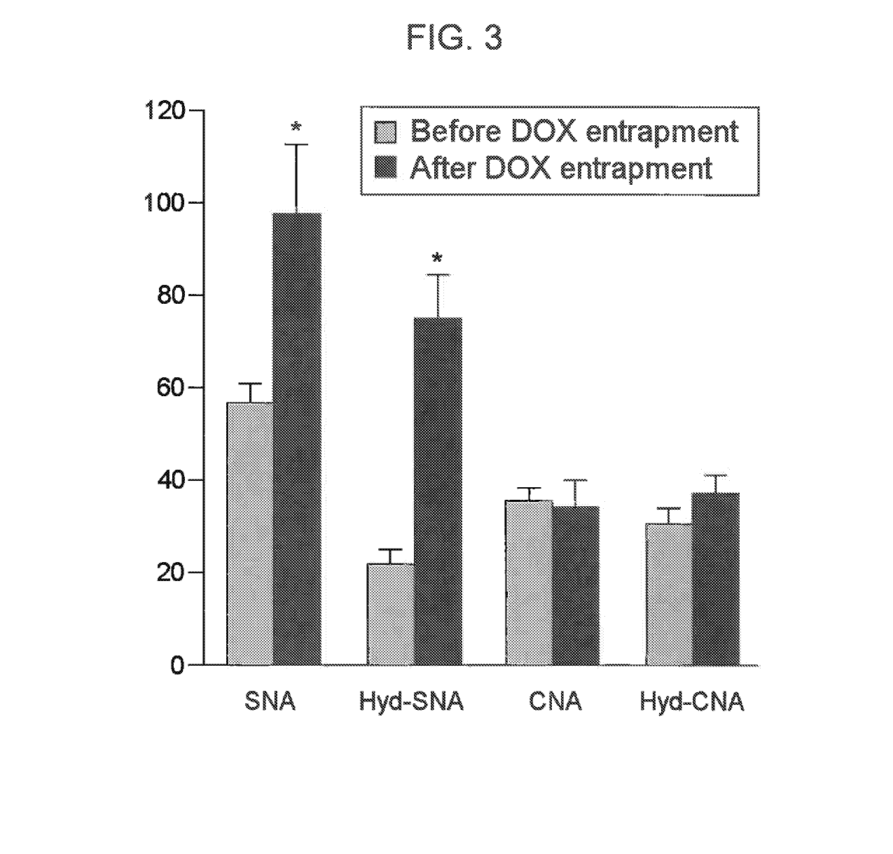

Particle sizes of nanoassemblies were subsequently determined by DLS. All particles were smaller than 60 nm before entrapping DOX (FIG. 3). However, SNA and Hyd-SNA, which are self-assembled nanoassemblies, increased in particle size after DOX entrapment. On the contrary, CNA and Hyd-CNA showed no statistically significant changes before and after loading DOX, and the particle sizes of these cross-linked nanoassemblies were smaller than 40 nm regardless of drug entrapment approaches (physical entrapment versus chemical conjugation). As for drug entrapment, SNA showed the highest DOX loading by 56 weight % (wt %), followed by Hyd-SNA (42 wt %), Hyd-CNA (37 wt %), and CNA (27 wt %). Despite the different particle size and drug entrapment yield, none of the particles showed agglomeration or precipitation in water.

DOX-loaded nanoassemblies with a cross-linked core (CNA and Hyd-CNA) enhanced particle stability during purification and storage in comparison to SNA and Hyd-SNA that interacted with ultrafiltration membrane when highly concentrated (>100 mg/mL) forming gel-like materials. No precipitation was observed under normal conditions handling nanoparticle solutions at concentrations lower than 20 mg/mL. Nanoassemblies readily went through 0.22 μm filters, and could be reconstituted in aqueous solutions (e.g. deionized water) after freeze drying. Despite enhanced particle stability and easy sterilization, we stored nanoassemblies as powder and prepared fresh samples for every experiment to avoid variability in quality of samples between batches. DOX concentrations were determined using filter-sterilized nanoassemblies.

### Drug Release Rates

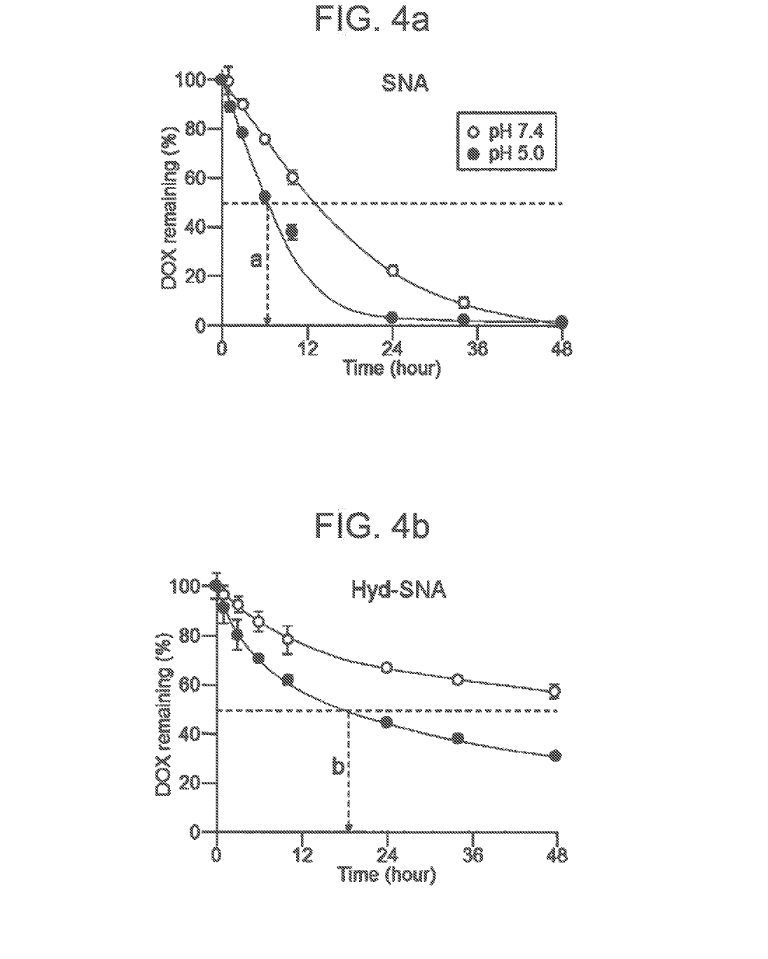

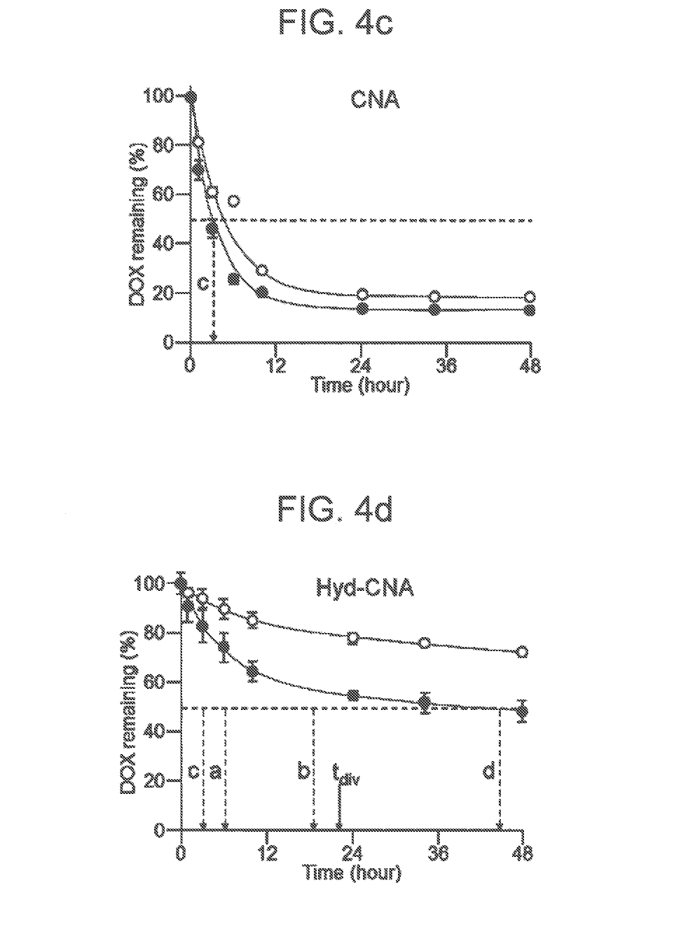

FIG. 4 shows DOX release patterns for SNA, Hyd-SNA, CNA, and Hyd-CNA. The determination of drug release half-life (t_{1/2}) at the physiological condition pH 7.4 was problematic because Hyd-CNA released drugs less than 50% in 72 hours (an incubation period we used for cytotoxicity assays). In addition, CNA released drugs quickly at pH 7.4, expanding the t_{1/2} range too broad to compare the effect of drug release rates on cytotoxicity of different nanoassemblies. All polymer nanoassemblies accelerated DOX release at pH 5.0 in comparison to pH 7.4, releasing more than 50% of drug entrapped in 48 hours. Our preliminary experiments showed that cell division time (t_{div}) for A549 cells was average 22 hours. Therefore, we calculated t_{1/2} at pH 5.0 rather than 7.4, which allowed us to determine t_{1/2} for all nanoassemblies within 48 hours while comparing conditions of t_{1/2}<t_{div} and t_{1/2}>t_{div} (denoted as Groups 1 and 2 in Table 1). Determining t_{1/2} at pH 5.0 is also clinically relevant because intracellular lysosomes (pH 4.8) and endosomes (pH 5.5 to 6.5) are acidic.

Accumulated amount of drugs released from nanoassemblies was also compared at pH 7.4 and 5.0 for 48 hours for each nanoparticle. As shown in Table 1 below, only nanoassemblies entrapping through covalent drug conjugation (Hyd-SNA and Hyd-CNA) showed a significant difference in acid-accelerated drug release, releasing approximately 24 to 27% more drugs at pH 5.0 as opposed to pH 7.4, whereas the total drugs released from SNA and CNA (DOX was entrapped through physical entrapment) appeared similar.

TABLE 1
Summary of data analyzed.

| | | Nanoassemblies ^{a} | | | |
|---|---|---|---|---|---|
| | Control | Group 1 | | | Group 2 |
| Samples | Free DOX | SNA | Hyd-SNA | CNA | Hyd-CNA |
| Drug release | N/A | 6.37 | 18.48 | 3.24 | 44.52 |
| half-life, | | | | | |
| t_{1/2}, (hour) ^{b} | | | | | |
| Accumulated | 100 | 99.14 | 42.49 | 80.26 | 27.90 |
| drug release | | | | | |
| at pH 7.4 | | | | | |
| in 48 h (%) ^{c} | | | | | |
| Accumulated | 100 | 98.28 | 69.10 | 89.27 | 51.50 |
| drug release | | | | | |
| at pH 5.0 | | | | | |
| in 48 h (%) ^{c} | | | | | |
| IC 50 (μM, | 0.53 ± 0.20 | 0.64 ± 0.57 | 0.86 ± 0.35 | 0.39 ± 0.08 | 2.65 ± 1.04 |
| DOX | | | | | |
| equivalent) | | | | | |
| Relative | 1.00 | 1.21 | 1.62 ^{d,}* | 0.74 | 5.00 ^{e,}* |
| cytotoxicity | | | | | |

^{a} Nanoassemblies were divided based on the drug release half-life (t_{1/2}) and cell division time (t_{div}): Group 1 (t_{1/2} < t_{div}) and Group 2 (t_{1/2} > t_{div}).
^{b} Drug release half-life was determined by plotting drug release-time curves in Figure 4.
^{c} Accumulated drug release was calculated from area under the drug release-time curve (AUC) in Figure 4, and normalized with respect to the initial drug loading (100%).
^{d} Data were significantly different in comparison to CNA.
^{e} Data were significantly different in comparison to Free DOX.

Interestingly, SNA released 98% of total DOX entrapped, yet CNA, released drugs at the fastest rate regardless of pHs, still entrapped 11 to 20% of DOX inside the particle. We confirmed that free DOX escaped completely from the dialysis bags in a few hours, and thus, 2% drugs in SNA as well as 11 to 20% DOX in CNA were considered being entrapped not in the dialysis bag but inside nanoassemblies.

### Cytotoxicity

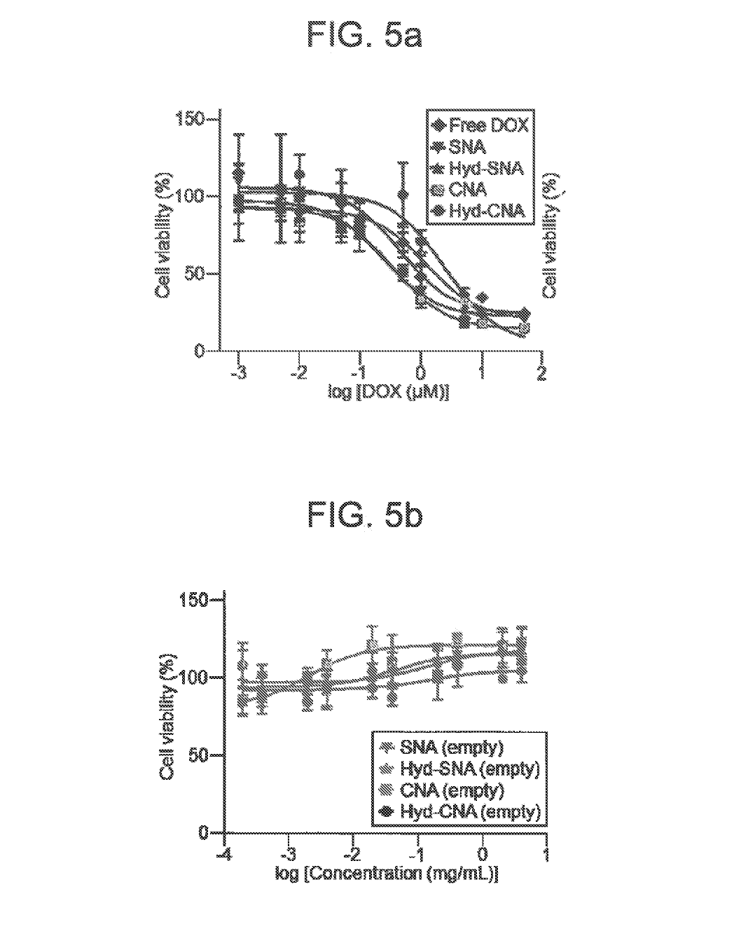

Biological activity of drug-loaded nanoassemblies was then evaluated by in vitro cytotoxicity assays, using free DOX and empty nanoassemblies as controls (Table 1 and FIG. 5). The cytotoxicity assays revealed that faster drug release did not always lead to greater cytotoxicity of nanoassemblies. CNA, which showed the shortest drug release half-life, was as potent as free DOX and SNA in terms of IC50 values (Table 1). SNA released drugs approximately 50% slower than CNA, t_{1/2}=6.37 versus 3.24 hours (a and c in FIG. 4), yet it was also similarly effective to kill A549 cancer cells compared with free DOX. As t_{1/2} becomes longer, nanoassemblies showed a lower IC50 value, and Hyd-SNA (t_{1/2}=18.38 h) was 2.2 fold less potent than CNA (t_{1/2}=3.24 h) based on IC50 values. Interestingly, our data demonstrate that the drug release rate (t_{1/2}) is not linearly proportional to cytotoxicity (IC50), but instead, t_{1/2} needs to be shorter than cell division time (t_{div}) for nanoassemblies to kill cancer cells effectively. It is noted that Hyd-CNA (t_{1/2}=44.52 and 51.50% drug released) showed the highest IC50 value (2.65 μM or 5 times less active than free DOX), considering that t_{div} of A549 cells was 22 hours and that Hyd-CNA has similar drug release half-life and drug release efficiency compared to Hyd-SNA. FIG. 5 shows dose-response curves of nanoassemblies with and without DOX, indicating that toxicity of empty drug carriers is negligible. These results suggest that the drug release rate might be the rate-determining factor for cytotoxicity of nanoassemblies because other particle properties such as particle size (<100 nm), stability (no precipitation over time), and surface properties (PEG coating) were similar among nanoassemblies. It is also surmised that fine-tuning of drug release with respect to cell division time could maximize drug efficacy by using drug molecules efficiently.

### DISCUSSION

Fast drug release is commonly expected to lead to greater drug efficacy for drug carriers. Such common expectation, however, does not explain how drug carriers can be equally, or even more, potent in comparison to free drug formulations in vivo. Enhanced drug delivery to tumors and improved bioavailability of drug are two major factors that also frequently explain greater therapeutic efficacy and low toxicity of drug-loaded drug carriers as opposed to free drugs. However, no drug carriers show the same therapeutic efficacy in vivo, even if the same drug payload was used. We also confirmed significant differences in in vivo antitumor activity and toxicity profiles of drug carriers, which can control drug release in response to stimuli or cell-specific receptors. Previous studies also pointed out that particle size, shape, and surface charge can be important as well to determine in vivo performance of drug carriers. Nevertheless, the answers to these questions remained difficult because each drug carrier employs a different approach to control drug release patterns, which affects aforementioned particle properties.

In this study, therefore, we prepared four types of particles, which have drug-binding linkers and cross-linkers in combination, by using self-assembling block copolymer micelles as a starting template. These four types of particles represent: 1) nanoparticle drug carriers coated with biocompatible PEG and loaded with anticancer drugs through physical entrapment (SNA); 2) nanoassemblies similar to SNA but entrapping drugs through a pH-labile linker that degrades in either extracellular tumoral or intracellular acidic condition (Hyd-SNA); 3) nanoassemblies based on SNA and cross-linked in the core for improved particle stability (CNA); and 4) nanoassemblies with improved particle stability and controlled drug release capability (Hyd-CNA).

Particle sizes of empty nanoassemblies were similar, ranging between 20˜60 nm (FIG. 3). Drug entrapment led to a considerable change in particle size, while SNA and Hyd-SNA doubled and tripled the particle size respectively. Considering that these two nanoassemblies are formed from self-assembling block copolymers, it is speculated that block copolymers rearranged during drug entrapment and produced larger particles, which are stable enough to retain spherical structure in water avoiding precipitation. SNA contained calcium salt and DOX, which would have stabilized the particles effectively, retaining particle size below 100 nm (clinically relevant to accumulate in tumors efficiently). It is likely that Hyd-SNA formed smaller particles than SNA because it has longer side chains modified with hydrazide groups that were further conjugated with drug payloads, which contributed together to form a compact core in the particle. In comparison to self-assembled particles, polymer cross-linked nanoassemblies retained particle size before and after drug entrapment. CNA, which loaded DOX through physical entrapment similarly to SNA, was as small as Hyd-CNA where DOX was conjugated through a hydrazone bond. It is noted that CNA and Hyd-CNA with cross-linked cores still showed efficient drug entrapment yield (>27 wt %), although that was approximately 10% lower than that of SNA or Hyd-SNA. The drug entrapment yield of Hyd-CNA (37 wt %), slightly higher than CNA (27 wt %), is likely attributed to that hydrazide drug binding linkers provide more space for DOX molecules to get inside the particle. These results demonstrate that four nanoassemblies with similar particle properties are successfully prepared.

Drug release patterns of the four nanoassemblies were distinctive enough to show varied drug release half-life. FIG. 4 indicates that nanoassemblies entrapping drugs through covalent chemical conjugation release drug more slowly than through physical entrapment (SNA versus Hyd-SNA or CNA versus Hyd-CNA). Cross-linking led to accelerated drug release in CNA, yet slowed down drug release from Hyd-CNA. Although both CNA and Hyd-CNA were mixed with DOX after cross-linking the cores, DOX molecules may be present at the vicinity rather than the center of the cross-linked core of CNA while they are likely entrapped in the core of Hyd-CNA. This hypothesis is supported partially by an increase, even though it is subtle, in particle size of Hyd-CNA following DOX entrapment in comparison to CNA (FIG. 3). FIG. 4 also shows that four types of nanoassemblies can be categorized by two nanoparticle groups according to the drug release half-life (t_{1/2}=a, b, c, and d) with respect to cell division time (t_{div} in FIG. 4, average 22 hours for A549 cells): 1) Group 1 with t_{1/2}<t_{div} (SNA, Hyd-SNA, and CNA); and 2) Group 2 with t_{1/2}>t_{div} (Hyd-CNA). Nanoassemblies, regardless of the composition, released DOX faster at pH 5.0 than pH 7.4, leading to an increase in total drug release. Nanoassemblies entrapping DOX through physical entrapment (SNA and CNA) released more than 80% of total drug in 48 hours, while Hyd-SNA and Hyd-CNA, nanoassemblies entrapping drugs through covalent chemical conjugation, led to 69.10% and 51.50% drug release at pH 5.0 in 48 hours (42.49% and 27.90% at pH 7.4), respectively.

Subsequent cell experiments revealed that fast drug release did not always result in greater cytotoxicity of nanoassemblies, evaluated by IC50 values, but that nanoassemblies with drug release half-life (t_{1/2}) shorter than cell division time (t_{div}) killed cells as effective as free drugs. The IC50 values and relative cytotoxicity of the nanoassemblies are summarized in Table 1, indicating that all nanoassemblies in Group 1 (SNA, Hyd-SNA, and CNA) were more effective to kill cells than Hyd-CNA (Group 2). Within in the same group, nanoassemblies that released drugs faster (t_{1/2}=c<a<b, FIG. 4) indeed showed lower IC50 values (IC50=c<a<b, Table 1), yet their relative cytotoxicity was within 2 fold in comparison to free DOX. On the contrary, the IC50 value of Hyd-CNA was 5 fold greater than free DOX, showing less anticancer efficacy. Although data are still limited to draw a general conclusion, these results suggest that drug release rates should be synchronized with cell division time to maximize drug efficacy, which may partially explain why drug carriers that release drugs slowly in vivo can still show effective antitumor activity in vivo. At the same time, it is expected that drug carriers that release drugs in a tunable manner would be effective to shrink tumors comprising cancer cells with different cell division times (e.g. fast-growing and quiescent cancer cells affected by heterogeneous oxygen supply, blood flow, interstitial pressure, and other tumor microenvironmental factors).

Cytotoxicity of a drug-loaded drug carrier is often attributed to toxicity of the empty drug carrier, which can behave as a surfactant to destabilize the cancer cell membrane or interacts with intracellular organelles (e.g. mitochondria, transporters, and enzymes), causing the programmed cell death (apoptosis). FIG. 5 confirmed that all empty nanoassemblies used in this study caused no toxicity at a concentration up to 10 mg/mL, which is clinically relevant to carry their drug payload at a therapeutic level in vivo (i.e. If drug loading is 10 wt %, a dose of 10 mg drug/kg can be achieved by injecting 1% drug solution with respect to a body weight of a patient as well as animal). Therefore, any additive cytotoxicity caused by nanoassemblies is negligible under experimental conditions in this study, considering the high DOX entrapment yield for each nanoparticle (SNA: 56 wt %; Hyd-SNA: 42 wt %; CNA: 27 wt %; and Hyd-CNA: 37 wt %). However, mechanisms of differential cellular responses to drug carriers with varied drug release rates should be detailed further in future studies because this study demonstrated only that fast drug release rates or high concentrations of drug released from nanoassemblies resulted in greater cytotoxicity, and furthermore, cell death is a complicated event that can be triggered by different reasons.

### CONCLUSION

Four types of nanoassemblies, modified through covalent drug-binding and core cross-linking in combination, were prepared in this study to identify the differences in physicochemical and biological properties of polymer nanoassembly drug carriers. Particle properties, such as particle size (<100 nm), drug entrapment yields (27 to 56 wt %), and surface property (PEG coating), were controlled similar between the nanoassemblies to avoid variables that could affect cytotoxicity. In comparison to an approach to cross-link the particle core, the covalent conjugating of drugs to nanoassemblies was a more effective way to increase the release half-life (t_{1/2}) for an anticancer drug (DOX), ranging between 3.24 and 44.52 h at the lysosomal condition (pH 5.0). In vitro cytotoxicity assays revealed that nanoassemblies (SNA, Hyd-SNA, and CNA) were equally effective as free DOX to kill cancer cells as long as the t_{1/2} (3.24 to 18.48 h) is shorter than the cell division time (t_{div}, 22 hours for A549). On the contrary, Hyd-CNA with t_{1/2}=44.52 h (>t_{div}) was 5 fold less effective in killing cells compared to free DOX or other nanoassemblies. Although further studies are needed, these results suggest that optimal drug release rates for nanoparticle drug carriers can be determined by controlling the drug release half-life with respect to cell division time to maximize therapeutic efficacy of anticancer drug payloads. In conclusion, nanoassemblies to which cross-linkers and drug-binding linkers are incorporated in combination would provide pharmaceutical advantages such as uniform particle size, physicochemical stability, fine-tunable drug release rates, and maximum cytotoxicity of entrapped drug payloads.

### Example 2

The chemical formula below demonstrates an example of a “single pot” preparation of a nanoassembly with (1) cross-linked block copolymers (acridine yellow, cross-linker), (2) a first active agent (IR820, imaging agent, hydrophobic) covalently bound to the block copolymers by a drug-binding linker and (3) a second active agent (Doxorubicin HCl, anticancer drug, hydrophilic) physically entrapped in the core. The single pot reaction sequence was that block copolymers reacted with the cross-linkers in DMSO (50˜100 mg polymer/mL DMSO, 50° C., overnight), followed by addition of the first active drug and second drug sequentially. The product was collected by dialysis against deionized water and freeze drying.

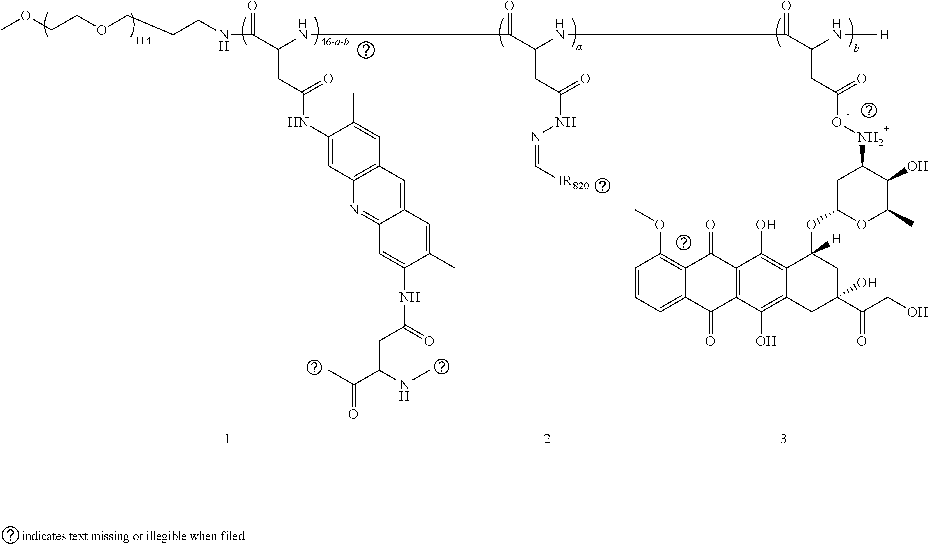

### Example 3

The chemical formula below demonstrates an example of preparing a nanoassembly including two active agents covalently bound to the block copolymers in the absence of any active agent physically entrapped by the block copolymers. The synthesis procedure to prepare such a nanoassembly is to (1) react the block copolymers with a cross-linker (adipic acid, 50˜100 mg polymer/mL DMSO, NHS:DIC:DMAP=2:2:0.2, 50° C., overnight), (2) conjugate a first active agent (Doxorubicin, anticancer drug) covalently to the drug-binding linker in the nanoassembly core by means of hydrazide bond, and (3) conjugate a second active agent (Wortmannin, anticancer drug) covalently to the block copolymers by a hydrazide drug-binding linker in the nanoassembly core, followed by dialysis against deionized water and freeze drying to collect the final product. The ratio of bound first active agent to bound second active agent is controlled by molar ratio of the two active agents used as starting materials.

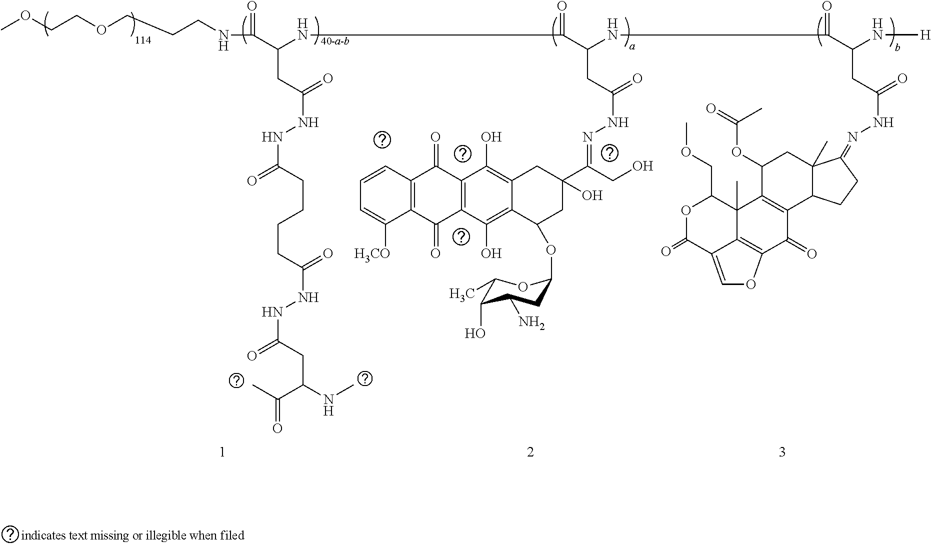

In summary, the biocompatible nanoassemblies and methods of making the same disclosed in this document provide a number of benefits and advantages. The nanoassemblies are particularly stable and of appropriate size for in vivo delivery to desired target sites in a mammal/human. The method of production allows one to make nanoassemblies with a customized core environment for hydrophobic, hydrophilic and ionizable active agents or guest molecules. Active agents and guest molecules may both be covalently bound and physically entrapped in the nanoassemblies while also allowing control of cross-linking yields. In this way it is possible to fine-tune drug release rates and thereby maximize efficacy.

The nanoassemblies are also suitable for wide-ranging applications including but not limited to: (a) drug delivery, protein delivery, gene delivery, imaging, remote hyperthermia, etc.; and (b) custom design and synthesis of drug carriers for specific drug analogues. The nanoassemblies exhibit the size, stability and biocompatibility characteristics necessary to achieve desired delivery performance for each such application.

The foregoing has been presented for purposes of illustration and description. It is not intended to be exhaustive or to limit the embodiments to the precise form disclosed. Obvious modifications and variations are possible in light of the above teachings. All such modifications and variations are within the scope of the appended claims when interpreted in accordance with the breadth to which they are fairly, legally and equitably entitled.

What is claimed:

1. A nanoassembly including a core protected by a biocompatible shell, comprising:
a plurality of cross-linked block copolymers including drug-binding linkers and block copolymer cross-linkers; and
a first active agent covalently conjugated to said plurality of cross-linked block copolymers by said drug-binding linkers.

2. The nanoassembly of claim 1 further including a second active agent physically entrapped in said core by said plurality of cross-linked block copolymers.

3. The nanoassembly of claim 2, wherein said nanoassembly has a diameter of 5 nm to 200 nm.

4. The nanoassembly of claim 2, wherein said nanoassembly has a cross-linking yield of 1 to 50%.

5. The nanoassembly of claim 2, wherein said nanoassembly has a drug loading of 1 to 60% by weight.

6. The nanoassembly of claim 2, wherein said drug-binding linkers include permanent linkers and degradable linkers.

7. The nanoassembly of claim 2, wherein said cross-linkers include permanent linkers and degradable linkers.

8. The nanoassembly of claim 2, wherein said drug-binding linkers are selected from a group consisting of (1) an aliphatic compound with amino, carboxyl, hydroxyl, ketone or thiol groups and cross-linking formed through amide, ester, carbamate, imine, hydrazone, and disulfide bonds (2) an aromatic compound with amino, carboxyl, hydroxyl, ketone or thiol groups and cross-linking formed through amide, ester, carbamate, imine, hydrazone, and disulfide bonds and (3) mixtures thereof.

9. The nanoassembly of claim 2, wherein said cross-linkers are selected from a group consisting of (1) an aliphatic compound with amino, carboxyl, hydroxyl, ketone or thiol groups and cross-linking formed through amide, ester, carbamate, imine, hydrazone, and disulfide bonds (2) an aromatic compound with amino, carboxyl, hydroxyl, ketone or thiol groups and cross-linking formed through amide, ester, carbamate, imine, hydrazone, and disulfide bonds and (3) mixtures thereof.

10. The nanoassembly of claim 2, wherein both said drug-binding linkers and said cross-linkers are selected from a group consisting of (1) an aliphatic compound with amino, carboxyl, hydroxyl, ketone or thiol groups and cross-linking formed through amide, ester, carbamate, imine, hydrazone, and disulfide bonds (2) an aromatic compound with amino, carboxyl, hydroxyl, ketone or thiol groups and cross-linking formed through amide, ester, carbamate, imine, hydrazone, and disulfide bonds and (3) mixtures thereof.

11. The nanoassembly of claim 2, including cross-linkers with pH degradable bonds.

12. The nanoassembly of claim 2, including cross-linkers with light degradable bonds.

13. The nanoassembly of claim 2, including cross-linkers with heat degradable bonds.

14. The nanoassembly of claim 2, including cross-linkers with bonds degradable by enzymatic activity.

15. The nanoassembly of claim 2, wherein said first active agent is selected from a group consisting of a diagnostic agent, an imaging agent, a therapeutic agent, multiple diagnostic agents, multiple imaging agents, multiple therapeutic agents and mixtures thereof.

16. The nanoassembly of claim 15, wherein said second active agent is selected from a group consisting of a diagnostic agent, an imaging agent, a therapeutic agent, multiple diagnostic agents, multiple imaging agents, multiple therapeutic agents and mixtures thereof.

17. The nanoassembly of claim 2, wherein said first active agent and second active agent are different.

18. The nanoassembly of claim 2, wherein at least one of said first active agent and said second active agent is selected from a group consisting of a small molecule having less than 1,000 atoms, a large molecule having at least 1,000 atoms, a peptide, plasmid DNA, siRNA, a fluorescent dye, a contrast agent and mixtures thereof.

19. The nanoassembly of claim 2, wherein said first active agent and second active agent include at least one hydrophobic agent and at least one hydrophilic agent in a single nanoassembly.

20. A method of making a biocompatible nanoassembly, comprising:
cross-linking block copolymers including drug-binding linkers and block-copolymer cross-linkers; and
covalently binding a first active agent to said block copolymers.

21. The method of claim 20 including physically entrapping a second active agent in said cross-linked block copolymers.

22. The method of claim 21 including providing a plurality of block copolymers with drug-binding linkers and block copolymer cross-linkers.

23. The method of claim 21 including dissolving (a) block copolymers with drug-binding linkers and block copolymer cross linkers, (b) first active agent and (c) second active agent together in a solvent and simultaneously completing said cross-linking, said covalent binding and said physical entrapping.

24. The method of claim 23, including simultaneously completing said cross-linking, covalent binding and physical entrapping while providing a cross-linking yield of between 1% and 50%, a drug loading of between 1% and 60% by weight and maintaining a nanoassembly having a diameter of 5 to 200 nm.

25. The method of claim 23 including ceasing further cross-linking when said nanoassembly reaches a most thermodynamically stable condition.

26. The method of claim 23 including no separate purification step.

27. The method of claim 21 including tuning said nanoassembly to entrap multiple active agents, control release profiles and achieve differential tissue-cell targeting in a controlled manner.
